(19) United States
(12) Patent Application Publication
(10) Pub. No.: US 2009/0324537 A1
(43) Pub. Date: Dec. 31, 2009

(54) Polymeric Materials as Stomach Filler and Their Preparation

(76) Inventors: Mircea Dan Bucevschi, Rehovot (IL); Monica Colt, Rehovot (IL); Mendy Axlerad, Rehovot (IL)

Correspondence Address: ELMORE PATENT LAW GROUP, PC, 515 Groton Road, Unit 1R, Westford, MA, 01886, US

(21) Appl. No.: 12/411,533

(22) Filed: Mar. 26, 2009

Related U.S. Application Data
(63) Continuation of application No. 12/295,517.
(60) Provisional application No. 60/787,166, filed on Mar. 30, 2006.

(51) Int. Cl. A61K 31/765; C08G 63/06; A61P 3/04

(52) U.S. Cl. 424/78.38; 528/271

(57) ABSTRACT

The present invention relates to swellable polymeric materials comprising a synthetic polymer, or copolymer, comprising a carboxylic group and a biopolymer that are suitable for bioapplications. Because of their ability to swell, the polymeric materials are suitable for use as stomach fillers for the treatment of being over weight or obese, or for inducing the feeling of being satiated. Methods for preparing the swellable polymeric materials comprising aqueous reaction systems are also disclosed.

### RELATED APPLICATIONS

This application is a continuation of U.S. application Ser. No. 12/295,517 filed Mar. 30, 2007, which is a US National stage entry of International Application No. PCT US07/065638, which designated the United States and was filed on Mar. 30, 2007, published in English, which claims the benefit of U.S. Provisional Application No. 60/787,166 filed Mar. 30, 2006.

### FIELD OF INVENTION

The present invention relates to synthetic carboxylic copolymers and polymeric composite materials obtained from binary mixtures of synthetic carboxylic copolymer and a biopolymer.

### BACKGROUND OF THE INVENTION

Obesity is a major medical problem affecting millions of people worldwide. In addition to the psychosocial stigmas associated with the condition or disease, many medical problems may develop. Hypertension, heart disease, diabetes, hyperlipidemia, degenerative arthritis and certain types of cancer are more common among overweight individuals. For those persons more than forty-five kilograms overweight, the risk of sudden premature death is twelve times higher than normal. Weight loss often results in significant risk reduction of these associated problems.

Medical studies dedicated to elucidate the cause and effect relation point to the following aspects.
a) The effect is caused by some organic anomaly in the gastrointestinal system (mouth, stomach, pancreas, small intestine, etc). Although alimentary consumption is normal or diminished, weight increases.
b) The effect has psycho-socio-economical causes (absence of moving, lack of will, state of nerves, stress and other), but organic functionality is normal. In these cases, increased weight is because of the consumption of a higher quantity of food.
c) The effect is the result of an inadequate diet based on food with high nutritive coefficients. In this situation, neuro-psychic dependence is generated which will manifest itself in an abnormal diet, tendency to eat a lot, and finally abnormal functionality.
d) The effect can have multiple causes (combination of the factors mentioned above).

The major public health problem owing to being overweight is the intensification of psycho, socio, and economical factors (which include inadequate diets due to the fast-foods industry). In principal, this means the gastrointestinal apparatus is generally healthy, and the base strategy for remediation of the situation should be to reduce food intake.

Several methods are known in the art to reduce food intake. These include surgical procedures, non-surgical procedures, low calorie intake formulas, pharmacological treatments, and “full stomach” principles.

Several surgical techniques have been tried which bypass the absorptive surface of the small intestine or aim to reduce stomach size by either partition or bypass. These procedures are both hazardous to perform in morbidly obese patients and fraught with numerous life-threatening postoperative complications. Moreover such operative procedures are often difficult to reverse.

Non-surgical approaches, including dietary, psychotherapy, medications and behavioral modification techniques, have yielded extremely poor results in multiple trials. However, they continue to be studied for possible improvements because they are more comfortable, less expensive, and preferred by patients compared to surgical techniques. The most popular non-surgical techniques involve dietary restrictions. Dietary restriction methods are well known in the art and aim to reduce food intake by suppressing appetite.

Low calorie formula methods are the most popular methods for weight loss and have their basis in “dietetic foods.” Dietetic foods represent edible compositions made from only natural products, synthetic food, or mixtures of natural products and synthetic food. A variety of such recipes are those exemplified in U.S. Pat. No. 5,063,073; U.S. Pat. No. 5,654,028; U.S. Pat. No. 6,426,077; U.S. Pat. No. 5,405,616; U.S. Pat. No. 6,103,269; U.S. Pat. No. 6,071,544; U.S. Pat. No. 6,468,988; U.S. Pat. No. 4,784,861; U.S. Pat. No. 6,020,324; U.S. Pat. No. 6,322,826 and U.S. Pat. No. 6,472,002. The concepts involved are heavy digesting vegetal fibers; food with collagen; and increasing the content of dietetic oils and the like. Major disadvantages of these methods are that they can be used only by a small number of patients whose metabolism can support the abnormal presence of some of the components; and the stationary time in the stomach is higher than normal causing patients to suffer gastrointestinal discomfort.

Pharmacological treatments involve substances, natural or synthetic, that are active in biochemical processes (at the endocrine and neurocrine levels) that suppress appetite. Treatments include a) increase the tone of the pyloric sphincter (see U.S. Pat. No. 5,760,082; U.S. Pat. No. 6,071,544; U.S. Pat. No. 6,426,077; and U.S. Pat. No. 6,468,988); b) controlling Cholecystokinin (CCK) levels (see U.S. Pat. No. 3,859,942; U.S. Pat. No. 5,795,895; U.S. Pat. No. 6,403,657; U.S. Pat. No. 6,468,962 and U.S. Pat. No. 6,475,530) and c) gene therapy (see U.S. Pat. No. 6,057,109 and U.S. Pat. No. 6,309,853). Patients receiving treatment for weight loss through medication frequently experience complications such as a cessation of performance of the medication due to a “nutritional deficiency.” Frequently it is difficult to predict which patients are likely to experience unacceptable results due to “nutritional deficiencies.”

“Full stomach” principle suppresses appetite by giving the sensation of satiety. The technique consists of ingesting some kind of “food” which induces stationary time extension in the stomach. Known strategies include: a) stomach filling with inflatable bag and tube combinations (see U.S. Pat. No. 3,046,988; U.S. Pat. No. 4,133,315; U.S. Pat. No. 4,246,893; U.S. Pat. No. 4,416,267; U.S. Pat. No. 4,899,747; U.S. Pat. No. 4,485,805 and U.S. Pat. No. 4,739,758) and b) stomach filling with hydrogels (see U.S. Pat. No. 5,336,486; U.S. Pat. No. 6,018,033; U.S. Pat. No. 5,750,585; and U.S. Pat. No. 6,271,278).

The full stomach principles based on hydrogels are well known in the art with different variations. Wounderlich J. C. et al. in U.S. Pat. No. 5,405,616 and U.S. Pat. No. 6,103,269 describe using a freeze dried blend of a polymeric mixture of gelatin or collagen hydrolysate, drugs, and auxiliaries for processing (i.e., plasticizers, odorants, etc.). The dried material in contact with the aqueous medium from the stomach swells in a few minutes and then dissolves to a solution capable of emptying from the gastrointestinal tract without problems.

Acharya R. N. in U.S. Pat. No. 5,336,486 discloses compositions based on polymers commercially available under the generic name “calcium polycarbophil” as lozenges that when ingested in controlled quantities suppress appetite and cause a feeling of satiation without causing undesirable side-effects. The United States Pharmacopeia, 1990 edition, United States Pharmacopeial Convention, Inc., Rockville, Md., at page 218, indicates that calcium polycarbophil is a calcium salt of cross-linked polyacrylic acid. Those who use this composition declared that appetite was reduced significantly and no feelings of hunger were present for several hours after taking the lozenges. It is not understood through what mechanism the results were achieved.

Chen Jun et al. in “Gastric retention properties of superporous hydrogel composite” J. Controlled Release, 64, 39-51, 2000, and in U.S. Pat. No. 6,018,033 and Park K. et al. in U.S. Pat. No. 5,750,585 and U.S. Pat. No. 6,271,278 disclose that hydrogels obtained by grafting and cross-linking a mixture of acrylic acid, acrylamide, potassium salt of 3-sulfopropyl acrylate and N,N′-methylenebisacrylamide in the presence of AcDi-Sol® (small cross-linked polysaccharide), swell in the stomach after oral administration and can be used as an auxiliary in diet control. These polymeric composites have the following disadvantages: a) the method of preparation does not permit integral conversion of monomers to polymer, resulting in contamination with toxic substances called “extractibles” which include non-reacted monomers, residues of initiators, and others. The deleterious effect of extractibles on patient comfort and health in traditional superabsorbent polymers is documented (see U.S. Pat. No. 5,075,344); and b) emptying the stomach by decreasing the gel phase means that the hydrogel's basic chemical structure remains intact in the small intestine. A possible second swelling would obstruct the small intestine and even the large intestine causing multiple, non-favorable implications.

Burnett D. R. et al. in WO 2004/056343 A1 discloses an ingestible formulation for transient, noninvasive reduction of gastric volume comprising polymeric formulations capable of being retained in the stomach for a certain period of time followed by rapid degradation upon entering an intestine. The dosage form of the polymeric formulations can be in the form of tablets, capsules, solutions, emulsions or suspensions. The polymeric formulations comprise a dehydrated combination of a cross-linked biocompatible polymer (e.g., an alginate) and a solubilizer/stabilization agent (e.g., xantan gum, propylene glycol alginate) and the like covered with an acid-sensitive coating (e.g., a gelatin). The formulations can also comprise active biological compounds used to treat being overweight or obesity, as well as other additives such as preservatives, sweeteners, colorants, flavorings, and the like. The cross-links are a polymer or copolymer of lactic acid, glycolic acid, trimethyl carbonate or any other hydrolysable ester which is susceptible to hydrolysis. The oral dosage forms and polymeric formulations typically achieve 90% of equilibrium swelling in about 6-18 hours, resulting in size increases of about 200%-1000%, and typically disappear from the gut in about 3-10 days. These polymeric formulations have the disadvantage that they act as a “false food” which must be continually ingested in large quantities to achieve a sensation of fullness. Also, the degree of swelling is small and the long stationery time in the body can result in accumulation of degraded products in organs other than the gastrointestinal tract, which can result in undesirable biological effects.

To prepare improved polymeric materials based on the “full stomach” principle, it is necessary to understand both bioprocesses active at the gastrointestinal tract level.

Gastrointestinal Tract

Normal physiology of the gastrointestinal tract covers the stomach and small intestine (Freita Jr., R. A., in “Nanomedicine”, Volume 1 Landes Bioscience Georgetown, Tex., 1999; Johnson L. R. in Physiology of the Gastrointestinal Tract, Raven Press, New York, 1981).

The stomach serves as a reservoir for food mixing, kneading and churning the solid food and regulating the emptying of its contents into the duodenum. The reservoir function involves temporary storage of ingested and secreted substances. Above a certain threshold volume, the stomach is “full” (whether the volume is large or small); i.e., the intragastric pressure increases very little with the addition of more food or fluid because the walls of the stomach relax to accommodate the load. The stomach also mixes ingested substances with gastric juice to dissolve and dilute food, knead solid materials to a particle size of less than 2 mm diameter, and finally, empty its contents into the duodenum slowly and in small volumes. The stomach is an irregularly pear-shaped bag (when the stomach is filled with food in a man standing erect, the stomach assumes an almost vertical position with a tubular shape), having a normal volume of approximately 1000 cm³ (up to 1500 cm³ in very large people, but as little as approximately 60 cm³ in newborns). However, 300-500 cm³ usually gives a person a sense of being full.

The inner wall of an empty stomach has longitudinal expansion pleats called rugae. As the stomach fills with food, the rugae flatten out and disappear, leaving an approximately 600 cm² smooth mucous membrane surface when the stomach is full. The uppermost epithelial layer of the stomach lining is the mucosa and is several millimeters thick. Almost all of the epithelial cells that line the surface are simple columnar mucous cells that secrete mucus. The gastric mucus is especially viscous, approximately 50-100 microns thick, and is highly resistant to both the digestive juices and the acid secreted by the stomach. The mucosa contains the secreting cells of the stomach arranged in small tubular units to form the gastric glands. Each day gastric glands secrete 1000-3000 cm³ of gastric juice. A residual of 50 cm³ is always present in the stomach, even after lengthy fasting. Secretion rates in young adults average 77 cm³/hr (male) and 70 cm³/hr (female) while fasting, 54 cm³/hr (male) and 38 cm³/hr (female) while sleeping, and 114 cm³/hr (male) and 99 cm³/hr (female) after eating. Gastric juice is an on average 1% aqueous solution with a specific gravity of approximately 1.006 (1.004-1.010) at pH ˜2.0. Besides water, the gastric juice also contains protein-digesting enzymes such as pepsin and rennin (chymosin), lipid-digesting enzymes such as lipase, hydrochloric acid, salt, mucous (glycoproteins) and regulatory peptides, such as gastrin and somatostatin. The acidity of gastric juice, expressed in mEq HCl, plays an essential part in food processing as a catalyst for enzymatic reactions (pepsin efficacy), both in terms of solubalizing alimentary components and in controlling protein digestion. Basal acid output refers to the quantity of hydrochloric acid secreted per hour by the stomach in the unstimulated basal state, expressed in milliequivalents of HCl per hour. The normal range is 1-5 mEq of HCl per hour. Maximal acid output refers to the total acid output during the hour after stimulation (i.e., after food intake) and has a normal range of 25-55 mEq HCl per hour.

The gastric motility (as a mechanical function) of the stomach is controlled centrally by local neurohormonal muscle. The muscle layers include the outer longitudinal, middle circular, and inner oblique fibers. Neuronal control involves the intrinsic myenteric plexus, the extrinsic postganglionic sympathetic fibers of the celiac plexus, and the preganglionic parasympathetic fibers of the vagus nerve. The vagal afferents are both relaxatory and excitatory. The time interval from food intake until emptying of stomach (also called gastric retention time-GRT), the stomach's content is subdued to an intragastric pressure (maximal stomach contraction pressure) of 5−15 kPa. The GRT ranges from 1 hour (liquid consistency, 10⁻³ Pas) to 12 hours (heavy paste consistency, 10⁵ Pas), with the average being 2-6 hours at an average consistency of 10³ Pas.

The emptying of the stomach is influenced by the substance, volume, osmolality and composition of the ingested meal. Liquids empty more rapidly than solids. The rate of gastric emptying is related to the square root of the volume, so that a constant proportion of the gastric contents empty per unit time. Stimulation of duodenal osmoreceptors with triglycerides, fatty acids or hydrochloric acid slows gastric emptying.

The action of stomach emptying is based on flow phenomenon of a liquid through an orifice. The orifice's width, which is much smaller than the vessel's width, in association with rheological characteristics of the fluid are critical factors that affect emptying speed (see Nielsen, L. E. in “Mechanical Properties of Polymers Composites”; Marcel Dekker: New York, 1974; Schramm G. A. in “A Practical Approach to Rheology and Rheometry” Karlsruhe, Germany: Gebrueder HAAKE GmbH, pp 17-18, 1994). The restriction in flow is regulated by the pyloric sphincter which behaves as a tap. The orifice opening (maximum value corresponding to a diameter of about 2 mm) is controlled both by stomach motility (i.e., pH of mixture) and by neurostimulator activity of the gastrointestinal tract.

The well-churned food mixture, now called chyme, is ejected through the pyloric valve into the duodenum of the small intestine. Nervous system and hormonal signals (e.g., enterogastrone) arising mainly from the duodenum, but also partly from the stomach control the degree of contraction of the pyloric sphincter and thereby control the rate at which the chyme is emptied from the stomach into the duodenum of the small intestine.

The small intestine is a continuous tube with three well-defined sections—the duodenum, jejunum, and ileum. The total length is commonly reported as approximately 7 meters, but this measurement is for tissues taken from cadavers which have lost all muscle tone. In the living body, the small intestine is only 3-5 meters in length. The small intestine extends from the pylorus of the stomach all the way to the large intestine and occupies the greater portion of the abdominal cavity. About 90% of all digestion and absorption takes place in the small intestine, including up to 6 liters/day of the 8-10 liters/day of water that flows into it from swallowed saliva, ingested water, the acid fluid secreted by the stomach, bile and pancreatic juice, as well as fluid secreted by the upper small bowel itself. Food is passed along by muscular contractions in waves known collectively as peristalsis, with waves progressing arhythmically for distances varying from 10-100 cm in length, and occasionally over the entire length of the small intestine. Food is also broken up by rhythmic segmentation contractions within the irregular peristaltic motions, which are ring like contractions of the circular muscle ranging in frequency from 10-30/minute, with higher rates at the upstream end of the bowel.

After leaving the stomach, food enters the part of the small intestine known as the duodenum which is arranged in a horseshoe shape around the head of the pancreas. The Brunner's glands are found only in the duodenum and their mucus-containing secretion has a pH of 5.8-7.6, a specific gravity of 1.01, and a highly variable cholesterol concentration of 3.61 (0-31.5)×10⁴ g/cm³. As the chyme passes through the duodenum, it is neutralized in preparation of digestion (i.e., pH-is modified from pH=2-2.8 to pH=8.5-9), and is subjected to biodegrading enzymes. In particular, the pancreatic juice contains pancreatin, a mixture of the three digestive enzymes: trypsin (which digests protein), lipase (which digests fat), and amylase (which digests starch). The specific gravity of the fluid is 1.008, mean viscosity is 1.6 mPas (up to 5.8 mPas in patients with chronic pancreatitis), and the pH is 7-8. Pancreatic juice flows upon signaling from the hormone secretin which is manufactured by the mucous membrane of the duodenum and which sends its message as soon as partially digested food enters from the stomach. Bile is a bitter, yellowish fluid that helps to emulsify and digest fats to hasten their absorption from the intestines, activate the pancreatic enzyme lipase, stimulate intestinal movements, and inhibit fermentation of the bowel contents. The specific gravity of bile is 0.998-1.062. Absolute viscosity ranges from 0.843-2.342 mPas, and pH averages 7.5 (6.2-8.5) for hepatic bile, and 6.0 (5.6-8.0) for gallbladder bile. Hepatic bile contains 1.7-5.2×10⁻⁴ g/cm³ sugars and 1.2 (0.8-1.7)×10⁻³ gm/cm³ cholesterol, while gallbladder bile contains 8×10⁻⁴ g/cm³ sugars and 6.3 (3.5-9.3)×10⁻³ gm/cm³ cholesterol, plus 0.33% lipids.

In the jejunum, fats, starches, and proteins are broken down to their smallest components and are absorbed by the cells lining the bowel. Of particular interest, absorption of sugars takes place chiefly in the upstream portion of the small intestine, specifically in the duodenum and upper jejunum. Hence the concentration of glucose in the chyme peaks and then sharply declines in the jejunum because starches of all molecular sizes are enzymatically reduced to the simplest sugars prior to absorption, although disaccharides are not as readily absorbed. Cholesterol is also absorbed mainly in the jejunum.

In the ileum, water is absorbed (˜0.07-0.40 cm 3/sec) along with calcium, other minerals, and vitamins (especially vitamin B₁₂). Bile is recaptured and returned to the liver via the hepatic portal vein and the lymphatic thoracic duct systems. Fat is also absorbed more rapidly in the ileum than in the duodenum or jejunum.

### Polymeric Hydrogels

Absorbent materials for water and aqueous media, including fluids secreted by the human body, are known. These materials are polymeric powders, granules, microparticles or fibers. Upon contact with aqueous systems, they swell by absorbing the liquid phase into their structure, without dissolving in it. A “hydrogel” is polymeric material after it has absorbed water. If the water absorbency is greater than 100 g water/g dried polymer the material is called “superabsorbent” polymer (SAP).

Hydrogels are used as drug carriers for orally administered pharmaceutics. “Loading” the drug into the hydrogel occurs during the preparation of product, and “unloading” occurs during and/or after its interaction with aqueous media. To increase drug efficacy, unloading must occur in certain locations of the gastrointestinal tract and in accordance with certain phenomenological laws of delivery. Oral administration of drugs generally makes use of two classes of hydrogels: a) functional in stomach, and b) functional in small intestine with preferential locations (oral cavity, duodenum and other).

The stomach produces gastric secretions that comprise water, hydrochloric acid, pepsin and mucus (polysaccharide biogel). This medium has a pH of 1-3 and manifests proteolytic activity owing to pepsin proteolytic enzymes. The small intestine provides an aqueous medium with a chemical composition more complex than gastric secretions. It is characterized by a pH of 5-9 and has biodegradative enzymatic activity on proteins and polysaccharides.

For hydrogels active in the stomach, it is necessary that the carrier be polymeric and swell in acid aqueous media, remain in the stomach for a certain period of time different than the normal physiological time of emptying, and to be easy eliminate after fulfilling the function for which it was administrated. Additionally, the hydrogel should not obstruct the tract, generate toxic secondary products, and or otherwise be harmful in any way.

For a polymeric carrier to have the above properties in gastric secretions more variables need to be solved. Swelling in acid media (pH of 1-3) has been achieved for non-ionic macromolecular structures, cationic polymeric matrixes, and anionic polymeric materials partially neutralized. Morita R., Honda R., Takahashi Y., “Development of oral controlled preparation, a PVA swelling controlled release system, SCRS. I. Design of SCRS and its release controlling factor”, J. Controlled Release, 63,297-304, 2000; Shalaby W. S. W., Blevins W. E., Park K., “In vitro and in vivo studies of enzyme-digestible hydrogels for oral drug delivery”, J. Controlled Release, 19, 289-296, 1992; Podual K., Doyle F. J., Pappas N. A., “Dynamic behavior of glucose oxidaze-containing microparticles of poly(ethylene glycol)-grafted cationic hydrogels in an environment of changing pH”, Biomaterials 21, 1439-1450, 2000; U.S. Pat. No. 5,352,448; and U.S. Pat. No. 5,674,495.

Hydrogel retention in the stomach has been controlled by more known methods: floating systems (see Deshpande A. A., Shah N. H., Rhodes C. T., Malick W., “Development of a novel controlled-release system for gastric retention”, Pharm, Res. 14, 815-819, 1997), swelling and expanding systems (see U.S. Pat. No. 4,434,153;U.S. Pat. No. 4,207,890), bioadhesive systems (see Hang Y., Leobandung W., Foss A., Peppas N. A. “Molecular aspects of muco- and bioadhesion: Treated structures and site-specific surfaces”, J. Controlled Release, 65, 63-71, 2000), modified-shape systems (see U.S. Pat. No. 4,735,804 and U.S. Pat. No. 4,767,627), high-density systems (U.S. Pat. No. 3,507,952) and other.

The classic hydrogels used as carriers for biologically active compounds do not have a swelling capacity large enough to be used as a dietetic using the “full stomach” principle. Additionally, one of the most important problems associated with using synthetic polymers medically is biocompatibility.

Biocompatibility is an accumulation of biochemical characteristics that a material possesses which makes possible its acceptance by living organisms (human, animals and plants) as an integral part of them, without having spontaneously or in time the manifestation of some repulsive or toxic phenomena that are inflammatory, infectious or otherwise (Black J., “Biological Performance of Materials: Fundamentals of Biocompatibility”, 2d ed. M. Dekker, N.Y., 1992).

The standards that have guided biocompatibility testing are the Tripartite Guidance; the International Organization for Standardization (ISO) 10993 standards, which are known as the Biological Evaluation of Medical Devices and remain under development internationally; and FDA Blue Book Memoranda.

Non-biocompatibility has two sources: 1) the polymer, and 2) the residual raw materials used in polymer synthesis (e.g., monomers, initiators, solvents and auxiliaries of polymerization, or auxiliaries of processing to form three-dimensional networks such as cross-linkers for surface treatments, solvents, and others).

Only a small number of carboxylated synthetic polymers are biocompatible. One example is the commercially available “EUDRAGIT” line of polymers which include acrylic acid copolymers; ethyl acrylic acid, and methacrylic acid. Breitkreutz, J. in “Leakage of enteric (Eudragit L)-coated dosage forms in simulated gastric juice in the presence of poly(ethylene glycol)”, Journal of Controlled Release 67: 79-88, 2000.

Certain copolymers based on maleic acid are biocompatible and have been used in the medical field. Sethi, N. et al. “Safety evaluation of a male injectable antifertility agent, styrene maleic anhydride copolymer, in rats”, Contraception 39:217-226 19895; Lohiya N. K et al. “Repeated vas occlusion and non-invasive reversal with styrene maleic anhydride for male contraception in langur monkeys”, Int. J. Androl; 23: 36-42, 2000; Ottenbrite, R. M. “Antitumor activity of polycarboxylic acid polymers”, J. Macromol. Sci. Chem., A22(5-7), 819-832, 1985; and Spiridon D. “Synthesis and Biocompatibility of Maleic Anhydryde Copolymers: 1. Maleic Anhydride—Vinyl Acetate, Maleic Anhydride—Methyl Methacrylate and Maleic Anhydride—Styrene”, Polymer International, 43, 175-181, 1997.

A polymer is more biocompatible the richer it is in an organism's biopolymers. Thus, the most biocompatible (even completely) polymers are those that contain collagenic biopolymers: native collagen, soluble collagen, gelatin, and collagen hydrolysates. Hoffman A. S., Daly C. H., “Biology of Collagen”, Viidik Vunst J. Eds., Academic Press New York, 1980; Ward A. G., Courts A., “The Science and Technology of Gelatin”, Academic Press N.Y., 1977 and U.S. Pat. No. 5,376,375; U.S. Pat. No. 5,292,802; U.S. Pat. No. 5,945,101; U.S. Pat. No. 6,071,447 and other.

Specific properties of these polymers result from choice of raw materials and processes of preparation known in the art. They are singular materials and/or composites based on ionic or non-ionic polymers. Examples include a) poly(acrylic acid) and acrylic acid copolymers obtained by copolymerization of mono and polyfunctional monomers, and composite materials thereof (see U.S. Pat. No. 3,926,891; U.S. Pat. No. 4,090,013; U.S. Pat. No. A117,184; U.S. Pat. No. 4,190,562; U.S. Pat. No. 4,654,039; U.S. Pat. No. 4,666,983; U.S. Pat. No. 4,808,637; U.S. Pat. No. 4,833,222; U.S. Pat. No. 5,118,719; U.S. Pat. No. 5,567,478; and U.S. Pat. No. 5,629,377); b) cross-linked starch by graft polymerization of acrylonitrile, bifunctional polymerization monomers, and composite materials thereof with other natural and/or synthetic polymers (see U.S. Pat. No. 3,935,099; U.S. Pat. No. 3,997,484; U.S. Pat. No. 4,076,663; U.S. Pat. No. 5,453,323; and U.S. Pat. No. 6,107,432); c) polyacrylamide, acrylamide copolymers, and composite materials thereof using cross-linking copolymerization methods (see U.S. Pat. No. 4,525,527; U.S. Pat. No. 4,654,039; U.S. Pat. No. 5,408,019; and U.S. Pat. No. 5,712,316); d) maleic anhydride copolymers and polymeric composites thereof (see U.S. Pat. No. 3,959,569; U.S. Pat. No. 3,980,663; U.S. Pat. No. 3,983,095; U.S. Pat. No. 4,389,513; U.S. Pat. No. 4,610,678; and U.S. Pat. No. 4,855,179), e) modified celluloses (see U.S. Pat. No. 4,959,341; U.S. Pat. No. 5,736,595; U.S. Pat. No. 5,847,031; U.S. Pat. No. 6,833,488 and WO2005/084724); f) poly(vinyl alcohol) and copolymers thereof (see U.S. Pat. No. 4,124,748, and Bo J. “Study on PVA Hydrogel Crosslinked by Epiclorohydrin”, J. Appl. Polym. Sci., 46, 783-786, 1992); and g) polyaspartates and copolymers thereof (see U.S. Pat. No. 5,284,936; U.S. Pat. No. 5,847,013).

Commercial products of SAP based on polyacrylates, polyacrylamides or starch have been used in hygienic care and agriculture but not in the dietary field.

To obtain high purity absorbent materials for aqueous media with potential applications in the pharmaceutical and/or medical field, three-dimensional polymeric configurations can be obtained by a) chemical methods: ionic and/or coordinative intercomplexing (see U.S. Pat. No. 4,570,629 and U.S. Pat. No. 5,153,174), cross-linking with oligomers or reactive polymers that have reactive groups with double bonds or rings (see U.S. Pat. No. 5,489,261 and U.S. Pat. No. 5,863,984); cross-linking with radiations (see U.S. Pat. No. RE33,997; U.S. Pat. No. 4,264,155; and U.S. Pat. No. 5,948,429); and b) physical methods: cross-linking with microwave (see U.S. Pat. No. 5,859,077; and U.S. Pat. No. 6,168,762); freezedrying (see U.S. Pat. Nos. 5,676,967; and 5,869,080); and dehydrothermo crosslinking (see U.S. Pat. No. 4,837,285; U.S. Pat. No. 4,950,485; and U.S. Pat. No. 4,971,954).

Dehydrothermo-crosslinking for obtaining three-dimensional structures eliminates the risk of toxicity produced by secondary reaction products or modification of reaction product in which new types of covalent, ionic, or coordinative bonds form. Moreover, compared to freeze-drying or cross-linking with microwave radiation, dehydrothermo-crosslinking offers much more possibilities to regulate the three-dimensional networks (see Scotchford C. A. et al. “Osteoblast responses of collagen-PVA bioartificial polymers in vitro: the effects of cross-linking method and collagen content” Biomaterials 19, 1-11, 1998; and Giunchedi P. et al., Biomaterials 19, 157-161, 1998). However, hydrogels based on collagenic biopolymers and obtained by dehydrothermo-crosslinking lack the absorption capacity of the present invention.

Presented herein is a new class of SAP materials which exhibit superior performance without the disadvantages of previous SAP materials. This new class of SAP materials is useful in the diet aids field.

### SUMMARY OF INVENTION

It is an object of the present invention to provide substantially improved, orally administered polymeric materials for the treatment of obesity or being overweight based on such methods as false satiety, appetite elimination, inhibition of some neural signals, modification of some biochemical process involved in assimilation, and others. Simultaneously, the polymeric material may provide some target synergetic effects.

Another object of the present invention is to provide a polymeric composite comprising two polymers. One is a synthetic polymer and the other is a biopolymer. Combination ratios for the two polymers are chosen to confer a digestible character through the presence of the biopolymer, and to not confer energetic significance (caloric content) through use of the synthetic polymer.

Another object of the present invention is to provide a polymeric material that does not induce toxic effect because the polymeric material comprises a three-dimensional network formed only by interactions between the polymers present in the composite (food grade and pharmaceutical grade polymers), without participation of other chemical components. The biocompatibility of the new product is assured also by the fact that the synthetic polymer after biodegradation has an average molecular mass which does not permit it to enter the blood system by specific absorption. The absence of absorption into the blood system confers to the synthetic polymer an inert character and it is eliminated from the body.

A further object of the present invention is to provide a new polymeric material for oral administration that together with one or two glasses of water swells in the stomach to produce a hydrogel that induces a sensation of satiety.

Another object of the present invention is to provide a polymeric material that behaves in the stomach in a similar manner to that of commonly consumed food. Thus, a few minutes after administration, the hydrogel reaches a consistency similar to an alimentary bolus. Then in time, because of gastric secretions, the hydrogel becomes a paste, similar to chyme, with a consistency that permits in the end an easy emptying of the stomach.

Another object of the present invention is to provide a polymeric material with a gastric retention time adjustable to a patient's anatomophysiological particularities and medical strategy adopted for the treatment of being over weight or obesity. The material may also be modified to deliver active biological compounds (e.g., pharmaceutical products to suppress appetite or inhibit neural signals . . . etc.).

Another object of the present invention is to provide a polymeric material that responds positively to the enzymatic system of the small intestine by inclusion of polypeptidic chains in the three-dimensional network of the polymeric material. The content of the proteinaceous material controls the rate of biodegradation. Reaching the small intestine, the biodegradation process ends with macromolecular fragments soluble in aqueous media for easy elimination from the body.

Yet another object of the present invention is to provide a method of preparing the polymeric material comprising an aqueous solution from which the solid phase is separated and dried by thermal treatment for stabilization of the polymeric composite's three-dimensional configuration. The method of preparing the new polymeric material is ecologically friendly (no pollutant raw materials, no generation of secondary products, and no pollutant wastes).

In one aspect, the present invention features a swellable polymeric material comprising a composite of a synthetic polymer and a biopolymer, wherein the synthetic polymer is a carboxylic containing copolymer.

In another embodiment, the polymeric material is a granular solid with a circumscribed equivalent diameter, O_{eq}, of not less than 0.2 mm and not greater than 2 mm. In another embodiment, the O_{eq} is between 0.4 mm and 1.5 mm.

In another embodiment, the swellable polymeric material is represented by the formula:

[(AB)⁽⁻⁾C⁽⁺⁾]W

wherein,

A represents a carboxylic containing copolymer;

B represents a biopolymer;

C represents a counterion; and

W represents water bound to the polymer.

In another embodiment, A comprises co-monomers M1 and M2 in ratio of 20:80 to 80:20. In another embodiment, A comprises co-monomers M1 and M2 in a ratio of 40:60 to 60:40.

In another embodiment, M1 comprises co-monomers maleic anhydride and maleic acid. In another embodiment, M1 comprises co-monomers itaconic anhydride and itaconic acid. In another embodiment, M1 comprises co-monomers citraconic anhydride and citraconic acid. In another embodiment, M1 comprises co-monomers 2-octenylsuccinic anhydride and 2-octenylsuccinic acid.

In another embodiment, M2 comprises an olefin. In another embodiment, M2 comprises a monoolefin. In another embodiment, M2 comprises ethylene, propene, isobutylene, styrene, alpha-methylstyrene, alkylated styrenes, ethylstyrene, tertbutylstyrene, vinyl-toluene, vinyl esters of saturated C₁-C₄-carboxylic acids, vinyl formate, vinyl acetate, vinyl propionate, alkyl vinyl ethers, ethyl vinyl ether, butyl vinyl ether, acrylate, methacrylate esters, 2-ethylhexyl acrylate, n-butyl acrylate, isobutyl acrylate, t-butyl acrylate, hexyl acrylate, n-butyl methacrylate, lauryl methacrylate, isodecyl methacrylate, conjugated diolefins, butadiene, isoprene, piperylene, allenes, allene, methyl allene, chloroallene, olefin halides, vinyl chloride, vinyl fluoride, polyfluoro-olefins, esters of monoethylenically unsaturated C₃-C₆-carboxylic acids, esters of monohydric C₁-C₈— alcohols and acrylic acid, esters of monohydric C₁-C₈-alcohols and methacrylic acid, esters of monohydric C₁-C₈-alcohols and maleic acid, mono esters of maleic acid, monomethyl maleate, 2-hydroxyethyl acrylate, hydroxypropyl acrylate, hydroxybutyl acrylate, hydroxyethyl methacrylate, hydroxypropyl methacrylate, hydroxybutyl methacrylate, N-vinyllactams, N-vinylpyrrolidone, N-vinylcaprolactam, acrylic and methacrylic esters of alkoxylated monohydric saturated alcohols, vinyl pyridine, vinyl morpholine, N-vinylformamide, dialkyldiallylammonium halides, dimethyldiallylammonium chloride, diethyldiallylammonium chloride, allylpiperidinium bromide, N-vinylimidazoles, N-vinylimidazole, 1-vinyl-2-methylimidazole, N-vinylimidazolines, N-vinylimidazoline, 1-vinyl-2-methylimidazoline, 1-vinyl-2-ethylimidazoline, 1-vinyl-2-propylimidazoline, acrylic acid, methacrylic acid, acrylamide, methacrylamide or acrylonitrile. In another embodiment, M2 comprises styrene.

In another embodiment, the ratio of M1:M2 is not less than 20:80 and not greater than 80:20. In another embodiment, the ratio of M1:M2 it not less than 40:60 and not greater than 60:40.

In another embodiment, M1 comprises co-monomers maleic anhydride and maleic acid, and M2 comprises styrene.

In another embodiment, B comprises a carbohydrate, protein, soybean protein, collagen, collagenic biopolymers, gelatin, collagen hydrolysates, or albumin casein. In another embodiment, B is a gelatin or a carbohydrate. In another embodiment, the gelatin is derived from either terrestrial or marine animals. In another embodiment, the carbohydrate is derived from vegetable sources. In another embodiment, B has a Bloom Index not less than 20 and not higher than 500 bloom. In another embodiment, B has a Bloom Index between 100 and 300 bloom. In another embodiment, B has an isoelectric point (IP) not less than 3.5 and not greater than 9.5. In another embodiment, B has an IP not less than 4.5 and not greater than 8.5.

In another embodiment, the ratio of A:B is from 95:5 to 55:45 by weight. In another embodiment, the ratio of A:B is from 90:10 to 70:30 by weight. In another embodiment, the ratio of A:B is 90:10, 85:15, 80:20, or 75:25 by weight.

In another embodiment, C is an inorganic cation. In another embodiment, C is Li⁽⁺⁾, Na⁽⁺⁾, K⁽⁺⁾, or NH₄⁽⁺⁾. In another embodiment, C is Na⁽⁺⁾ or NH₄⁽⁺⁾.

In another embodiment, the molar content of C (+), expressed in mol/gram of (A+B), is not less than 0.002 mol/g and not greater than 0.004 mol/g. In another embodiment, the molar content of C (+), expressed in mol/gram of (A+B), is not less than 0.0025 mol/g and not greater than 0.0035 mol/g.

In another embodiment, M1 comprises co-monomers maleic anhydride and maleic acid, M2 comprises styrene, B is a gelatin, C is Na⁽⁺⁾ or NH₄⁽⁺⁾.

In another embodiment, the ratio of A:B is from 95:5 to 55:45 by weight. In another embodiment, the ratio of A:B is from 90:10 to 70:30 by weight. In another embodiment, the ratio of A:B is 90:10, 85:15, 80:20, or 75:25 by weight.

In another embodiment, the polymeric material has humidity content not less than 1% and not greater than 15% by weight. In another embodiment, the polymeric material has a humidity content between 5% and 10% by weight.

In another embodiment, the viscozimetric average molecular mass, My, is not less than 100,000 and not greater than 2,500,000 evaluated from intrinsic viscosity, [η], in tetrahydrofuran at 25° C. In another embodiment, My is not less than 1,000,000 and not greater than 2,000,000 evaluated from intrinsic viscosity, [η], in tetrahydrofuran at 25° C.

In another embodiment, the free absorbency for distillated water, FADW, at 37° C. after 24 hours of contact with water is not less than 200 g/g. In another embodiment, FADW at 37° C. after 24 hours of contact with water is higher than 250 g/g.

In another embodiment, the acid binding capacity, ABC, in mEq HCl/g of polymeric material, is not less than 0.002 mEq HCl/g. In another embodiment, ABC, in mEq HCl/g of polymeric material, is higher than 0.0025 mEq HCl/g.

In another embodiment, the swelling phenomenon occurs not more than 30 minutes after oral administration in a subject. In another embodiment, the swelling phenomenon occurs not less than 30 seconds and not more than 10 minutes after oral administration in a subject. In another embodiment, the swelling phenomenon occurs not less than 1 minute and not more than 5 minutes after oral administration in a subject.

In another embodiment, the time from oral administration of the swellable polymeric material to the perceived sensation of fullness in a subject is not greater than 30 minutes. In another embodiment, the time from oral administration of the swellable polymeric material to the perceived sensation of fullness in a subject is not greater than 15 minutes.

In another embodiment, the time from oral administration of the swellable polymeric material to a subject to the start of stomach emptying is not less than 50 minutes. In another embodiment, the time from oral administration of the swellable polymeric material to a subject to the start of stomach emptying is not greater than 300 minutes. In another embodiment, the time from oral administration of the swellable polymeric material to a subject to the start of stomach emptying is 80 minutes to 200 minutes. In another embodiment, after oral administration of the swellable polymeric material the pressure exerted at the start of stomach emptying is not greater than 5 Pa. In another embodiment, after oral administration of the swellable polymeric material the pressure exerted at the start of stomach emptying is less than 1 Pa. In another embodiment, the swellable polymeric material has the same rheological properties as ground food.

In another aspect, the present invention relates to a composition comprising the swellable polymeric material of the present invention. In another embodiment, the composition further comprises a pharmaceutical carrier. In another embodiment, the composition is in the form of a tablet, capsule, caplet, pill, or elixir. In another embodiment, the present invention relates to a medicament comprising any of the swellable polymeric materials or compositions of the present invention.

In another aspect, the present invention relates to a method of treating being over weight or obese comprising administering to a subject in need thereof an effective amount of the swellable polymeric material of the present invention. In another embodiment, the method further comprises administering another form of treatment. In another embodiment, the treatment is gene therapy, surgical interventions, or administering an appetite suppressant.

In another aspect, the present invention relates to a method of inducing the feeling of satiety in a subject comprising administering to a subject in need thereof an effective amount of the swellable polymeric material of the present invention. In another embodiment, the swellable polymeric material takes the place of a meal. In another embodiment, the amount of swellable polymeric material is not less than 2 grams and not more than 20 grams. In another embodiment, the amount of swellable polymeric material is not less than 5 grams and not greater than 15 grams. In another embodiment, the swellable polymeric material is administered with water. In another embodiment, the amount of water is not less than 100 ml and not greater than 600 ml. In another embodiment, the amount of water is not less than 200 ml and not greater than 400 ml.

In another aspect, the present invention relates to a method of preparing a swellable polymeric material capable of inducing the sensation of satiety upon ingestion comprising: a) preparing an aqueous mixture of a synthetic copolymer comprising carboxylic groups; b) preparing an aqueous solution of an inorganic salt; c) preparing an aqueous mixture of a biopolymer; d) mixing the synthetic polymer mixture from step a) with the inorganic salt solution from step b) to form synthetic polymer-inorganic salt mixture; e) adding the biopolymer mixture from step c) to the synthetic polymer-inorganic salt mixture of step d) to form an aqueous mixture of the polymeric material; f) drying the polymeric material from step e); and g) thermally crosslinking the polymeric material of step f) to form the swellable polymeric material.

In another embodiment, the aqueous mixture of the synthetic copolymer and aqueous solution of the inorganic salt are mixed at a temperature not less than 20° C. and not greater than 90° C. In another embodiment, the aqueous mixture of the synthetic copolymer and aqueous solution of the inorganic salt are mixed at not less than 40° C. and not greater than 70° C. In another embodiment, the aqueous mixture of the synthetic copolymer and aqueous solution of the inorganic salt are mixed for not less than 1 hour and not greater than 4 hours. In another embodiment, the aqueous mixture of the synthetic copolymer and aqueous solution of the inorganic salt are mixed for not less than 2 hour and not greater than 3 hours.

In another embodiment, the aqueous mixture of the biopolymer is preheated to about 50° C. In another embodiment, the synthetic polymer-inorganic salt mixture and biopolymer mixture are mixed at about 50° C. In another embodiment, the synthetic polymer-inorganic salt mixture and biopolymer mixture are mixed for not less than 1 hour and not more than 4 hours. In another embodiment, the synthetic polymer-inorganic salt mixture and biopolymer mixture are mixed for not less than 2 hour and not more than 3 hours.

In another embodiment, the polymeric material is dried by hot air currents not less than 40° C. and not greater than 100° C. In another embodiment, the polymeric material is dried by hot air currents not less than 50° C. and not greater than 90° C. In another embodiment, the polymeric material after drying has a humidity content of 5-10% by weight.

In another embodiment, the polymeric material is thermally crosslinked at a temperature not less than 100° C. and not greater than 130° C. In another embodiment, the polymeric material is thermally crosslinked at a temperature not less than 105° C. and not greater than 125° C. In another embodiment, the polymeric material is thermally crosslinked for not less than 30 minutes and not greater than 4 hours. In another embodiment, the polymeric material is thermally crosslinked for not less than 1 hour and not greater than 3 hours. In another embodiment, the thermally crosslinked polymeric material is allowed to sit for 24 hours at room temperature.

In another embodiment, all mixing is done in a kneader.

These embodiments of the present invention, other embodiments, and their features and characteristics, will be apparent from the description, drawings and claims that follow.

### BRIEF DESCRIPTION OF THE DRAWINGS

FIG. 1 depicts polymer-polymer covalent intercoupling, between the synthetic polymer SMAC and the gelatin biopolymer.

FIG. 2 depicts a working concept of PMSF in the stomach and small intestine.

FIG. 3 depicts the evolution of the main properties of the PMSF in the stomach together with characteristic measurements associated with the “Full-stomach principle.”

FIG. 4 depicts device of piston type for swelling profiling, in which: 1-nylon cloth of 100 mesh or PE foil; 2-rubber ring; 3-polyethylene cylinder; 4-the piston packing rubber.

FIG. 5 depicts rheological tests of Oscillation Frequency Sweep with variation of stronger module (G′) depending on frequency and fitting the experimental data for finding the value of gel rigidity (E).

FIG. 6 depicts graphic representation of a conductometric titration of a PMSF sample with a 0.2N HCl solution together with the graphic method for evaluating the Acid Binding Capacity (AcBC) index.

FIG. 7 depicts oscillation stress sweep rheological tests of the variation of storage module (G′) and of loss module (G″) together with the processing mode experimental data for determining critical stress, (ic), for PMSF materials of the present invention.

FIG. 8 depicts oscillation time sweep rheological tests of the variation of storage module (G′) and of loss module (G″) together with the processing mode of experimental data for determining “t_{bio}” for PMSF materials of the present invention.

FIG. 9 depicts diagram of indexes' variation: (DLA)ₜ; [θ_{c}]ₜ and (φ_{eq})t, depending on time for finished products PMSF-1 and PMSF-5, corresponding to Example 1 and Example 5.s.

FIG. 10 depicts a graph showing the same rheological properties between a PMSF of the present invention, PMSF-1, and ground food (“BigMac-1” and “BigMac-2”). BigMac-1=BigMac (200 g)+Chips (150 g)+Mineral Water (200 mL)+simulated gastric fluid (50 mL); BigMac-2=BigMac (200 g)+Chips (150 g)+Mineral Water (400 mL)+simulated gastric fluid (50 mL).

### DETAILED DESCRIPTION OF THE INVENTION

The polymeric material as stomach filler (PMSF) is a composite polymer material useful as a diet aid in correlation with the “full stomach principle.” The PMSF of the present invention may be used for weight control and/or obesity treatment. They possess a macromolecular configuration of three-dimensional networks stabilized by covalent bonds. More precisely, the present invention relates to stomach filler materials for oral administration which swell in the stomach's aqueous media, filling the stomach, and giving a sensation of false satiety. In particular, the present invention relates to SAP composite materials which after emptying from the stomach degrade via biochemical processes in the small intestine from three-dimensional networks to linear chains, which are easy to eliminate from the gastrointestinal tract.

In one embodiment, the PMSF of the present invention is a granular solid that has a circumscribed diameter equivalent, abbreviated as “Deq”, not less than 0.2 mm and not greater than 2 mm. In another embodiment, the Deq is between 0.4 mm and 1.5 mm.

In one embodiment, the PMSF of the present invention has a chemical structure expressed by the formula:

[(AB)⁽⁻⁾∥C⁽⁺⁾]W

wherein:

[(AB)⁽⁻⁾II C⁽⁺⁾] is a polymeric substance comprising anionic salifiable polymers; and

W is water bonded to the polymeric substance and in equilibrium with the general humidity.

The term “polymeric substance” refers to polymeric materials based on their chemical structure.

The term “anion polymer composite” is defined by the following. The term “polymer composite” refers to a polymeric substance that a) is formed from two polymers with different macromolecular chemical structure called “polymer A” and “polymer B”; and b) the resulting composite, (AB), is a unique entity that does not separate spontaneously to its components during application. This definition conforms with the accepted definition for polymeric composite materials. Gaylord, N. G. “Copolymers, Polyblends and Composites” Adv chem. 142, 76, 1975; Paul D. R. et al. “Polymer Blends”, Academic Press, New York 1978; and Manson J. A. et al. “Polymer Blends and Composites”, Plenum Press, N.Y., 1976. It is understood that the term “composite material” may include other substances such as drugs, stimulators, inhibitors, odorants, emollients, plasticizer and others, as a particular application warrants. These type of composite materials when used in the diet area are generically referred to as a “special combination.”

The term “anionic” refers to a polymeric composite (AB) generating in aqueous media a negative electrochemical potential as the result of the presence in its structure of some free acid functional groups capable of dissociating into anions.

The term “salifiable” refers to saline linkages between univalent inorganic cations, symbolized as “C(+)”, and the free anionic groups of anionic polymeric composite.

The symbol “∥” denotes the saline chemical bond (salt type) between anionic and cationic groups.

In one embodiment, the PMSF of the present invention has a humidity content not less than 1% and not more than 15%. In another embodiment, the humidity content is between 5% and 10% by weight.

The chemical composition of PMSF in the dry state (without humidity of equilibrium) is characterized by
an A:B ratio ranging from A:B=55:45 to A:B=95:5, expressed in weight percent. In an another embodiment the ratio ranges from A:B=70:30 to A:B=90:10 weight percent;
the molar content of C⁽⁺⁾, expressed in mol/gram of (A+B), ranging from not less than 0.002 mol/g to not higher than 0.004 mol/g. In another embodiment, the molar content ranges between 0.0025 mol/g and 0.0035 mol/g (A+B).

In one embodiment, Polymer A is a synthetic copolymer. Synthetic copolymers may be prepared in a single stage, such as free radical polymerization, or in two stages, polymerization followed by chemical modification (known as “polymer-analogous transformations”).

In one embodiment, Polymer A is a binary copolymer comprising monomers M1 and M2 at a ratio M1:M2 of not less than 20:80 and not greater than 80:20. In another embodiment, the ratio is between 40:60 and 60:40. In another embodiment, M1 is a co-monomer comprising a functional group which upon contact with water confers an acid character. In another embodiment, M1 comprise anhydride and a polymerizable acid, such as maleic anhydride, itaconic anhydride, citraconic anhydride, 2-octenylsuccinic anhydride and, respectively, the corresponding acids resulted by hydrolysis of anhydride groups (maleic acid, itaconic acid . . . etc.). In one embodiment, M1 comprises maleic anhydride (“MAH”) and maleic acid (“MAC”).

Co-monomer M2 is any type of substance that from a thermodynamic point of view performs the condition to give reactions of copolymerization with co-monomer M1. In one embodiment, M2 are radical polymerization monomers that do not possess free chemical groups. In one embodiment, M2 monomers are monoolefins such as ethylene, propene, isobutylene, styrene, alpha-methylstyrene, and alkylated styrenes such as ethylstyrene or tertbutylstyrene, vinyl-toluene, vinyl esters of saturated C₁-C₄-carboxylic acids such as vinyl formate, vinyl acetate or vinyl propionate, alkyl vinyl ethers with at least 2 carbon atoms in the alkyl group, such as ethyl vinyl ether or butyl vinyl ether, acrylate or methacrylate esters such as 2-ethylhexyl acrylate, n-butyl acrylate, isobutyl acrylate, t-butyl acrylate, hexyl acrylate, n-butyl methacrylate, lauryl methacrylate and isodecyl methacrylate; conjugated diolefins such as butadiene, isoprene, and piperylene; allenes such as allene, methyl allene and chloroallene; olefin halides such as vinyl chloride, vinyl fluoride and polyfluoro-olefins, esters of monoethylenically unsaturated C₃-C₆-carboxylic acids, i.e. esters of monohydric C₁-C₈-alcohols and acrylic acid, methacrylic acid or maleic acid, monoesters of maleic acid, i.e. monomethyl maleate, and hydroxyalkyl esters of said monoethylenically unsaturated carboxylic acids, i.e. 2-hydroxyethyl acrylate, hydroxypropyl acrylate, hydroxybutyl acrylate, hydroxyethyl methacrylate, hydroxypropyl methacrylate and hydroxybutyl methacrylate, N-vinyllactams such as N-vinylpyrrolidone or N-vinylcaprolactam, acrylic and methacrylic esters of alkoxylated monohydric saturated alcohols, vinyl pyridine and vinyl morpholine, N-vinylformamide, dialkyldiallylammonium halides such as dimethyldiallylammonium chloride, diethyldiallylammonium chloride, allylpiperidinium bromide, N-vinylimidazoles such as Nvinylimidazole, 1-vinyl-2-methylimidazole and N-vinylimidazolines such as N-vinylimidazoline, 1-vinyl-2-methylimidazoline, 1-vinyl-2-ethylimidazoline or 1-vinyl-2-propylimidazoline, acrylic acid, methacrylic acid, acrylamide, methacrylamide or acrylonitryl. In one embodiment, M2 is styrene (“S”).

In one embodiment, PMSF of the present invention is poly(styrene-co maleic acid) in acid form (without cations), referred to as “SMAC.” SMAC may be obtained with high chemical purity from poly(styrene-co maleic anhydride), “SMAH”, prepared by any process known in art. In one embodiment, SMAH is prepared by mass polymerization.

In one embodiment, SMAC comprises copolymers with the following structural characteristics:
molar co-monomeric compositions, expressed as S:MAC, ranging from 1:1 to 3:1, still further, S:MAC=1:1; and a content of free ester groups of less than 0.5 molar percent.
viscozimetric average molecular mass, My, not less than 100,000 and not greater than 2,500,000, still further, My is between 1,000,000 and 2,000,000; and intrinsic viscosity [il], in tetrahydrofuran solution at 25° C. of not less than 0.3 dl/g and not greater than 2 dl/g, still further, il is between 0.5 dl/g and 2.1 dl/g.

Polymer B represents biopolymers. A non-limiting example of a biopolymer that may be used in the present invention is a protein of animal origins or carbohydrate of vegetable origins which are easily digestible in the gastro-intestinal tract. In another embodiment, the biopolymer may be proteins commonly used in the pharmaceutical industry such as: collagen and collagenic biopolymers such as gelatin and collagen hydrolysates, albumin casein, and soybean protein. In a further embodiment, the biopolymer is food grade or pharmaceutical grade gelatin obtained from skin, bones, tendons, or other types of conjunctive tissue from different animals. The Bloom Index for these gelatins is not less than 20 and not higher than 500 bloom. In one embodiment, the Bloom Index is between 100 and 300 bloom and the isoelectric point (IP) is not less than 3.5 and not greater than 9.5. In one embodiment, the IP=4.5-8.5.

Substance C represents cations. More precisely, C represents univalent inorganic cations such as Li⁽⁺⁾; Na⁽⁺⁾; K⁽⁺⁾ or NH₄⁽⁺⁾ obtained from LiOH; NaOH; KOH and NH₄OH. In one embodiment, C is Na⁽⁺⁾ or NH₄⁽⁺⁾. The corresponding inorganic compounds (e.g. NaOH, NH₄OH . . . etc.) are called “alkaline agents.”

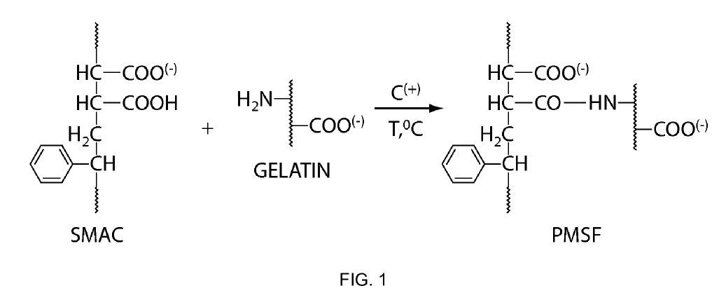

The PMSF of the present invention have three-dimensional networks generated and stabilized by polymer-polymer interactions conducive to forming covalent cross-linking bonds during the preparation of polymeric composite. The chemical reaction is depicted in FIG. 1.

The PMSF of the present invention comprise SAP characterized by the following parameters:
Free absorbency for distillated water, FADW, at 37° C. after 24 hours of contact between the PMSF and water of not less than 200 g of water/g of PMSF. In a further embodiment, absorbency is greater than 250 g/g;
Gel rigidity, E, of the hydrogel swelled with distilled water after 24 hours at 37° C. of not less than 1 kPa. In a further embodiment, gel rigidity is greater than 2 kPa as evaluated by oscillation frequency sweep data techniques as described in “Test methods”;
Acid Binding Capacity, ABC, in mEq HCl/g of PMSF, of not less than 0.002 mEq HCl/g. In a further embodiment, ABC is greater than 0.0025 mEq HCl/g.

In one embodiment, the PMSF of the present invention is administrated orally in materials known to protect the active product from the aqueous media in the mouth and esophagus. The oral dosage form may be in the form of pharmaceutical capsules of gelatin, cookies, sticks, cakes and the like.

Even though the PMSF of the present invention are primarily used for treatments based on the “full stomach” principle, they are not limited to this dietary concept. They may also be used in treatments based on chemical appetite suppressant, gene therapy, and others.

In one embodiment, the PMSF of the present invention is used to treat patients who, physiologically, have gastrointestinal tracts with the following parameters:
gastric stomach volume between 300 cm³ to 1500 cm³ with 900 cm³ as the average volume;
sensation of fullness at 250 cm³ to 750 cm³ with 300-500 cm³ as the average value;
free acidity in the stomach before PMSF intake of 2 mEq HCl to 8 mEq HCl with 5 mEq HCl as the average value;
gastric juice secretion of 30 ml/hour to 120 ml/hour with 75 ml/hour as the average value;
gastric juice composition expressed as:
   hydrochloric acid content from 70 mEq/Liter to 100 mEq/Liter with 85 mEq/Liter as the average value;
   pepsin concentrations from 1 g/Liter to 5 g/Liter with 3 g/Liter as the average value;
intragastric pressure from 5 kPa to 15 kPa with 10 kPa as the average value;
gastric retention times from 1 hours to 6 hours with 3 hours as the average value;
pancreatic juice secretions from 30 ml/hour to 70 ml/hour with 50 ml/hour as the average value;
pancreatin concentrations from 2 g/Liter to 18 g/Liter with 10 g/Liter as the average value;
small intestine (duodenum+jejunum+ileum) retention times from 1 hours to 5 hours with 4 hours as the average value.

The PMSF of the present invention are generally used to replace the normal food corresponding to one or two or three meals. The PMSF “meal” is composed of the PMSF and water, but may contain other components such as, for example, “light food” which includes different drugs in accord with adopted medical protocols for the treatment of overweight and/or obesity. The term “normal food” has used herein refers to a mixture formed from solid and liquid materials.

The amount of PMSF administrated to replace one normal meal depends on physiological parameters of the gastrointestinal tract of the patient and on the medical characteristics of the adopted protocol for treatment. Generally the amount is not less than 2 grams and not more than 20 grams. In one embodiment, the amount of PMSF is between 5 grams and 15 grams.

The quantity of water administrated with the PMSF to activate the full stomach principle correlates with the water content in the stomach before administration but is generally not less than 100 ml of water and not more than 600 ml water. In one embodiment, the amount of water is between 200 ml water and 400 ml water.

The term “water” refers to an aqueous, non-alcoholic beverage with a salt concentration not higher than 3 g/liter. In one embodiment, the salt concentration is less than 1.5 g/liter with a pH not less than 3 and not greater than 9. In one embodiment, the pH is between 5 and 7. Generally, these parameters describe municipal tap water, mineral water without carbon dioxide, and the like. The term can also include distilled or carbonated water.

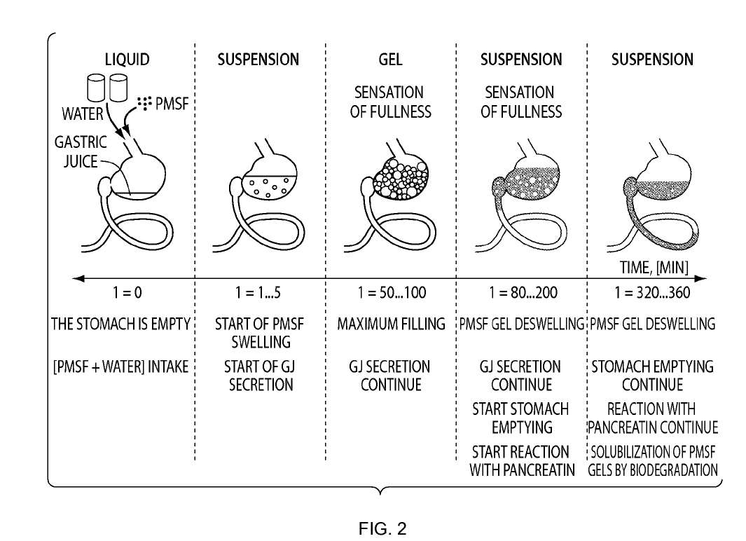

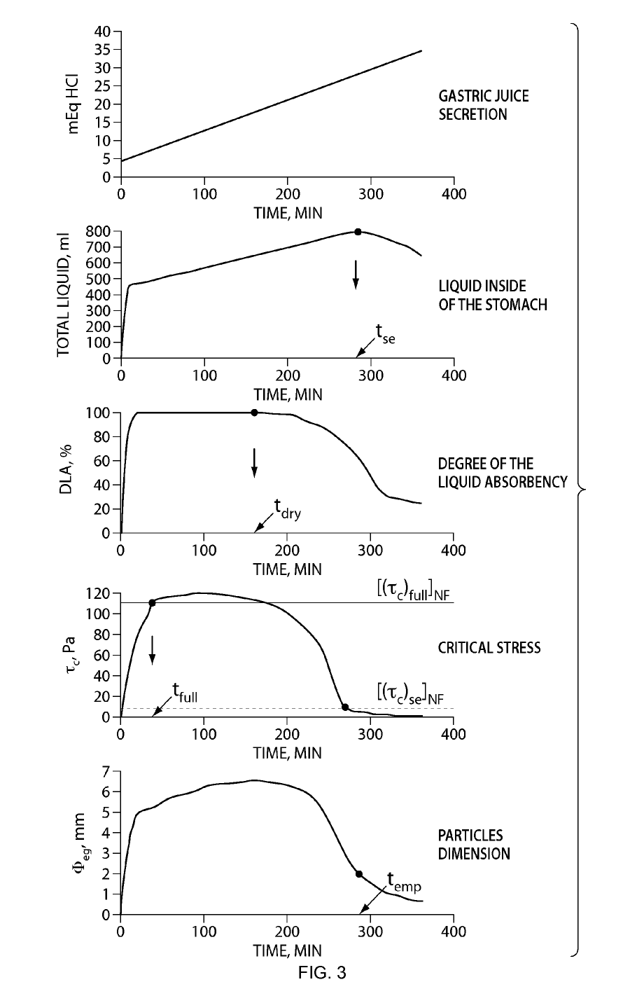

The PMSF of the present invention works in correlation with the full-stomach principle as depicted in FIG. 2. Variations of the characteristics and parameters are presented in FIG. 3.

As depicted in FIG. 2, PMSF of the present invention is orally administered together with a specified quantity of water. Upon contact with gastric juices from the stomach, a solid-liquid suspension forms which gradually transforms to a gel as the solid phase swells. Swelling occurs during an interval of time not less than 30 seconds and not greater than 10 minutes. In one embodiment, swelling occurs between 1 and 5 minutes after administration, taking into consideration the time necessary for separation of the material from PMSF packing. PMSF swelling continues concomitantly with activation of gastric juice secretion until the polymeric solid is transformed into a hydrogel which will be referred to as an “artificial bolus.”

Transformation of PMSF, into an “artificial bolus” by absorption of gastric solution from the stomach is characterized by
rate of absorbency, expressed as the time necessary for transformation of suspension into a gel, t_{gel}, expressed in seconds, is less than 60 seconds and not greater than 300 seconds. In one embodiment, t_{gel} is between 90 seconds and 180 seconds;
fullness time, which represent the time elapsed from administration of PMSF until the sensation of fullness of stomach is perceived, t_{full}, expressed in minutes, is not greater than 30 minutes. In one embodiment, t_{full} is less than 15 minutes.

The artificial bolus represents a material called a “dry gel” because a free liquid phase in between the gel's particles is not present. It can be eliminated mechanically by pulling small or medium pressures of the order 1−10 kPa (for example by suction with a vacuum of 300-600 mbar).

The dry gel is characterized by:

“fullness critical stress”, [τ_{c}]_{full}, in [kPa], at time t_{full}, corresponding to a “gel-sol” rheological transition of the material in the stomach (which correlates to the flow capacity of a system). It is evaluated using oscillation stress sweep techniques, and has values not less than 10 Pa. In one embodiment the values are higher than 25 Pa.

The value of the “fullness critical stress”, [τ_{c}]_{full} is adopted by medical protocol used for treatment of overweight and/or obesity, versus alimentary composition, called “normal food”, NF, for which the patient perceive the sensation of the fullness, and has a critical stress symbolized as [[τ_{c}]_{full NF}.

The dry gel in the artificial bolus is maintained for an interval of time, t_{dry}, of not less than 30 minutes. In one embodiment, t_{dry} is more than 60 minutes and less than 100 minutes under conditions of gastric juice secretions.

The dry gel, after t_{dry}, is transformed to “artificial chyme”, which represents a suspension gel particles and liquid. The liquid volume is formed from the volume corresponding to a supplementary secretion of gastric juice and the volume of water solution liberated from the gel particles during de-swelling. The de-swelling is associated with diminishing of gel particle dimensions.

The artificial chyme continues to confer sensation of fullness for an interval of time called time of beginning of stomach emptying, “tₛₑ”, measured from the administration of PMSF. In one embodiment, tₛₑ is not less than 50 minutes. In another embodiment, tₛₑ is greater than 80 minutes and less than 200 minutes. The material presents a critical stress which starts the stomach emptying, [τ_{c}]ₛₑ, not greater than 5 Pa.

In another embodiment, [τ_{c}]ₛₑ is less than 1 Pa.

[τ_{c}]ₛₑ adopted by medical protocol for the treatment of overweight and/obese is compared to an alimentary composition called “normal food”, NF, which starts the emptying of the stomach (from clinical test realized on patients) and has a critical stress value [[τ_{c}]ₛₑ]_{NF}.

Artificial chyme contains gel particles which provide a certain mass fraction having an average diameter of less than 2 mm resulting from both the deswelling phenomenon and stomach motility. When critical stress [τ_{c}]ₛₑ is reached but the material does not possess gel particles with diameters less than 2 mm, only liquid will be evacuated from the stomach because transfer of the gel is mechanically blocked.

PMSF assures a gastric retention time, GRT, defined by the relationship:

GRT=tₑₘₚ−t_{full}

wherein tₑₘₚ is the time for emptying and is not less than 90 minutes. In another embodiment, tₑₘₚ is from 120 minutes to 360 minutes from the administration of PMSF, corresponding to the situation where the dimension of all gel particles in the artificial chyme is less than 2 mm.

From the beginning of stomach emptying when the artificial chyme enters the duodenum, an intense process of degradation by pancreatic juice and bile occurs that ends with transformation of gel particles to a polymer solution.

Sensitivity of artificial chyme to enzymatic attack is reflected in the biodegradation time, “t_{bio}”, necessary to transform the material from the gel state to the solution state. The polymer solution proceeds through the rest of gastrointestinal tract and is eliminated from organism without enter in the sanguine circuit.

### Preparation of PMSF

The chemical composition of PMSF in the dry state represented by A, B and C presented above is used to calculate the quantities of raw materials necessary. M_{A}, M_{B}, and M_{c} are expressed in mass units [g or kg] and are used to prepare a quantity of finished product M_{PMSF} in g or kg.

In one embodiment, the PMSF of the present invention is prepared according to the following general procedures:

Aqueous Mixture Preparation [ABC-sol]

The raw materials, M_{A}, M_{B} and M_{c}, are treated with a quantity of water, M_{w} [g or kg], to form an aqueous mixture called [ABC-Sol]_{core}, with a content of solids, “cₛ”, not less than 5% and not greater than 4.5% by weight. In another embodiment, cₛ=15-35%.

Half of the necessary quantity of water, M_{w}, is used to prepare a solution of C⁺, called “SOL-C”, by direct dissolution of the corresponding alkaline agent in available water. The rest of M_{w} is used to prepare the biopolymer solution, called “SOL-B.” To a kneader equipped with a heating-cooling mantle, a quantity of synthetic polymer, M_{A}, and SOL-C are mixed at a temperature not less than 20° C. and not greater than 90° C. In another embodiment, the temperature is between 40° C. and 70° C. for not less than 1 hour and not greater than 4 hours. In another embodiment, the period of time is between 2 hours and 3 hours. SOL-B, pre-heated at temperature of 50° C., is added to the mixture. The mixing continues at the same temperature for an interval of time not less than 1 hour and not more than 4 hours. In another embodiment, the time is between 2 and 3 hours. [ABC-sol] is obtained as a mixture in the form of a viscous fluid with a consistency similar to a polymeric melt.

Obtaining [ABC-Dry] by Profiling and Drying [ABC-sol]

[ABC-sol] from above is cooled at a temperature not less than 15° C. and not greater than 55° C. In another embodiment the temperature is between 25° C. and 45° C. The viscous fluid is removed from kneader by extrusion through a stainless steal holed plate with holes having a diameter not less than 2 mm and not greater than 10 mm. In another embodiment, the holes are between 4 mm and 8 mm. The cylindrical shaped material is generally not less than 5 mm and not greater than 25 mm. In another embodiment, the length is between 10 mm and 15 mm. The cylindrical pieces of material are discharged on a metallic frame covered with a stainless steal wire net having holes of about 250 microns. The frame carrying the material is introduced in an oven with circulating hot air to eliminate the excess water by evaporation. The hot air current is not less than 40° C. and not greater than 100° C. In another embodiment, the temperature is between 50° C. and 90° C. The time of drying is adjusted so that at the end of the process the solid material has a humidity content of 5-10%.

The resulting dried material is ground in a cone mill. Afterwards, the ground material is separated by sieving (with vibrating sieves) into two solid fractions: one corresponding to [ABC-dry], and the other having geometrical characteristics that are appropriate for other applications called [ABC-rec]. Grading fractions that not correspond to a desired application, [ABC-rec]_{core}, are collected for re-processing.

Obtaining of PMSF Gross by Thermal Cross-Linking of [ABC-Dry]

[ABC-dry] is cross-linked thermally. The granular [ABC-dry] is distributed evenly on the surface of a stainless steal tray and the assemble is introduced in a laboratory oven with pre-hot air at a temperature, T, not less than 100° C. and not greater than 130° C. In another embodiment, the temperature is between 105° C. and 125° C. [ABC-dry] is maintain at these temperatures for an interval of time, t₁, not less than 30 minutes and not greater than 4 hours. In another embodiment, the amount of time is between 1 hour and 3 hours. The granular mass, referred to as PMFS-gross is taken out of the oven, cooled to 40-45° C., collected, and stocked in polyethylene vessels with hermetical seals for 24 hours at room temperature (20° C.-30° C.).

Thermal Consolidation of the Surface of PMSF-Gross

This operation is optionally a function of use of the finished product. For example, a particular use may require specific values for rate of absorbency, fullness time, fullness critical stress, dry gel time, critical stress at the start the stomach emptying, gastric retention time, and biodegradation time in small intestine.

After maturation PMSF-gross is re-introduced into the same laboratory oven mentioned above, with pre-hot air at a temperature not less than 120° C. and not greater than 160° C. In another embodiment, the temperature is between 125° C. and 155° C. The PMSF-gross is maintained at these temperatures for not less than 5 minutes and not more than 30 minutes. In another embodiment, the time period is between 10 and 25 minutes. The granular mass, which now represents the end product PMSF is taken out of the oven, cooled to 40-45° C., collected, and stocked in polyethylene vessels with hermetical seals at room temperature (20° C.-30° C.).

Recycling of Polymeric Composite [ABC-Rec]

The dried polymeric composite, [ABC-rec], is re-introduced into the kneader and a quantity of de-mineralized water (MW) is added so as to achieve the same solids content, “S”, and is mixed at a temperature not less than 20° C. and not greater than 90° C. In another embodiment, the temperature is between 40° C. and 70° C. Mixing is carried out at these temperatures for 2 hours. At this point [ABC-rec] is re-formed as [ABC-sol] which is further processed according to the procedure described previously.

The finished product, PMSF, resulting from the process described above is suitable for use in the diet area. It may be packed in, for example, capsules of gelatin, or as an alimentary product as the application suggests, using the methodologies of dosing and packaging known in the art.

### FADW (Free Absorbency in Distillated Water)

100 ml of distillated water is added to 3 beakers of 150 ml capacity. The beakers are then placed in a thermostatic water bath adjusted to 37° C. and are maintained at this temperature for 30 minutes. In each beaker is added 0.2±0.01 g of PMSF (M_{PMSF}) with known humidity determined with moisture analyzer Boeco SMO 01 (Germany) so that the granules are poured in the middle of the liquid's surface without stirring afterwards. Each beaker is covered with parafilm foil and is placed again in the thermostatic water bath for 24 hours. The content of each beaker is then added quantitatively to a 100 ml filter funnel with a filtering medium made from sintered glass with a porosity of 2 (pores with dimension between 40 . . . 100 μm) and tared on a technical balance. The funnel filter that contains the gel is filtered under vacuum at 500 mbar. After 2 minutes of vacuum action, the system is returned to atmospheric pressure and the funnel filter is weighed on the technical balance. The resulting mass of gel, mgel, is used to calculate swelling capacity (absorbency) in conformity with the formula below.

$$FADW = \frac{{m}_{gel} - {m}_{dry}}{{m}_{dry}} , [ g \text{/} g ]$$

### Gel Rigidity Determination

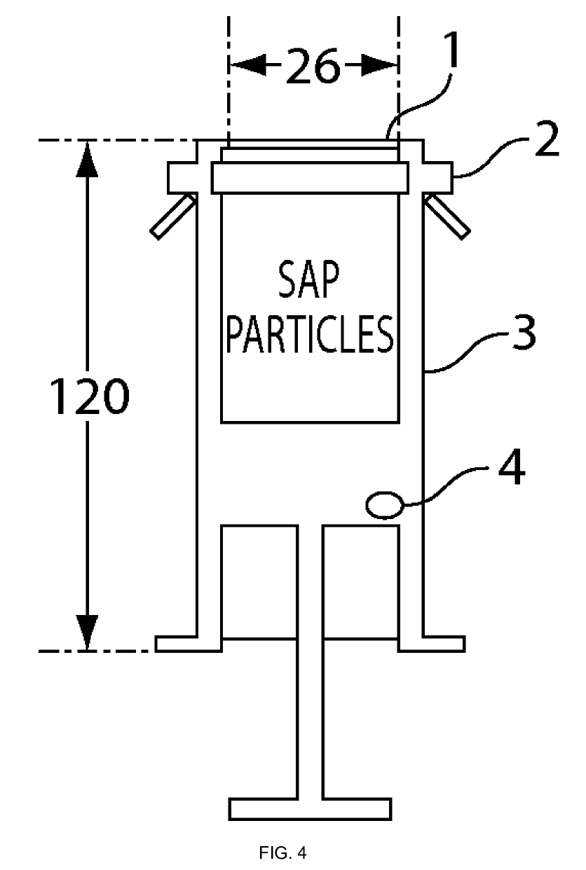

Gel rigidity, E, has been evaluated from rheological experiments using Oscillation Frequency Sweep techniques using a RheoStress 1 rheometer from ThermoHaake with a plate-plate sensor. About 5 grams of PMSF as gels resulting from the free absorbency test in distillated water are placed in a device depicted in FIG. 4.

A foil of polyethylene 1 covers the mass of PMSF gel at the upper part of cylinder 3 and is fixed with rubber ring 2 which prevents drying of the hydrogel by water evaporation. Piston 4 is moved until the layer of hydrogel is in contact with polyethylene foil 1. The piston is rotated 180° in a vertical plane after the rubber ring 2 and the polyethylene foil 1 are removed. Piston 4 is pressed until a cylinder of hydrogel 5 mm thick extrudes from the device. With a knife sections of the cylinder are cut into 5 mm thick discs. The disc is placed in the middle of the fixed plate of the sensor component of the plate-plate rheometer. The mobile plate of the sensor is moved over the sample until the distance between the two plates is 7 mm. The Oscilation Frequency Sweep rheological tests were conducted with plate-plate sensor system model PP35. All experiments were made in the frequency domain f—0.1±100 Hz, at 37° C. The experimental data was analyzed with software RheoWinPro of ThermoHaake. The experimental points corresponding to the G′ curve were fitted in connection with the rheological model (Rodol A. B., Cooper-White I., Dunstan D. E., Boger D. V.—in “Gel point studies for chemically modified biopolymer networks using small amplitude oscillatory rheometry” Polymer, 42, 2001, 185198).

G′=E+K*f^{q}

where:

E=gel rigidity, [kPa]
G′(ω)=storage modulus, [kPa]
f=oscillation frequency, [Hz]
K, q=material constants
From fitting the points to the curve a value for gel rigidity, E, is calculated.

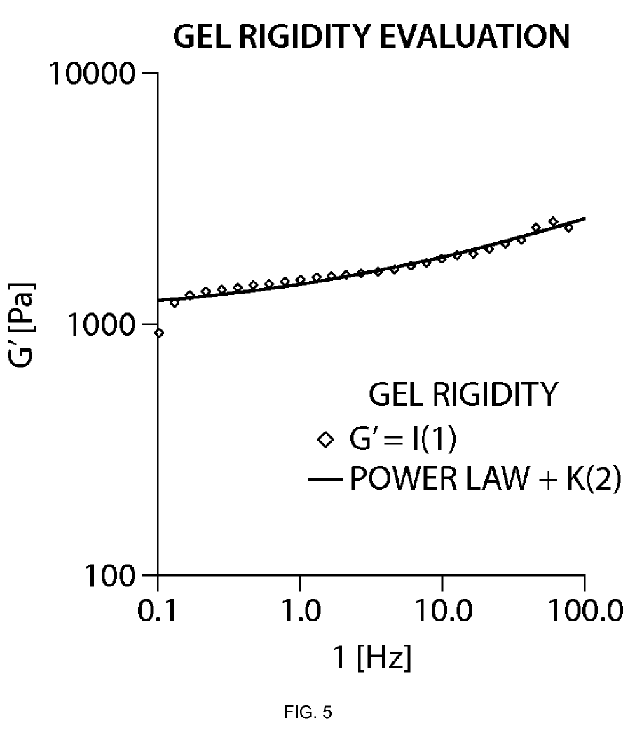

FIG. 5 depicts the graphic process for converting the rheological experimental data to the gel rigidity value.

### Acid Binding Capacity

Acid binding capacity, AcBC, was evaluated by conductometric titration using a JENWAY-Conductivity & pH meter model 4330, and Automatic Titrator, model 718 stat TITRINO (from Metrom-Switzerland).

0.5 grams of dried PMSF with a humidity content of u=5-10% and 50 ml of a 2% NaCl solution is placed in a 150 ml beaker. The beaker is covered with parafilm foil, and the content is stirred at room temperature with a magnetic stirrer for 2 hours. After removing the paraffin foil, electrodes for conductivity and pH measurements from JENWAY, and the dosing from automatic titrator TITRINO are introduced. The TITRINO titrator has in its alimentary vessel a 200 mEq/Liter of HCl (solution with titra THCI) solution. 60 ml of the HCl solution is added over 60 minutes. Every 60 seconds, conductivity, pH values, and volume of dosed HCl solution are collected. The volume and conductivity values are graphed. The changing point of the gradient of the variation's direction of conductivity versus HCl volume, determined by the intersection of the corresponding fitting line, gives the quantity of HCl volume consumed by PMSF, referred to as N_{HCI}. AcBC is then calculated by the relation

$$AcBC = \frac{{N}_{HCl} ⋆ {T}_{CHl}}{{m}_{d} ⋆ ( 1 - \frac{w}{100} )} , [ mEqHCl \text{/} gPMSF ]$$

where
N_{HCI}—volume of hydrochloric acid consumed at titration, [ml]
T_{HCI}—solution's concentration of hydrochloric acid used for titration, [mEq/ml]

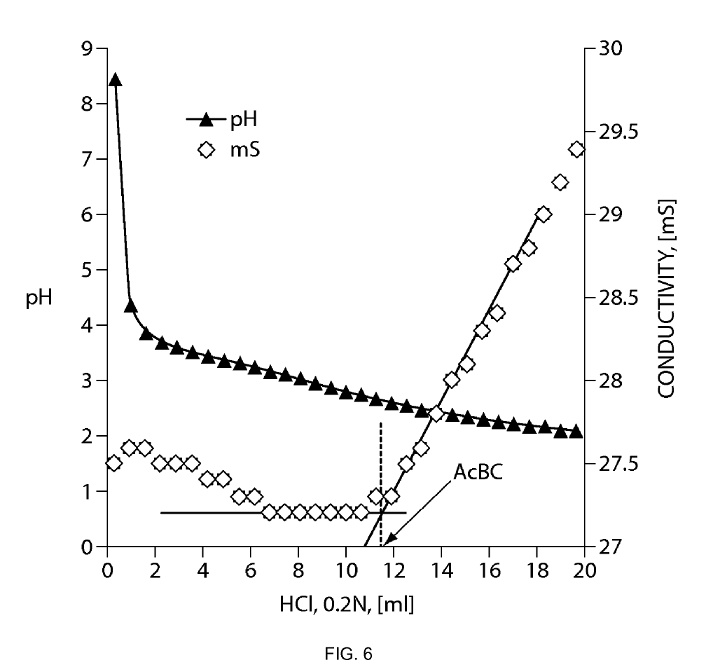

The graphic process of determining AcBC based on conductometric experimental data is exemplified in FIG. 6.

### Rate of Absorbency

The absorbency rate of PMSF, expressed as time, t_{gel}, is calculated by the method described in U.S. Pat. No. 4,587,308. In a 100 ml beaker were placed 50 ml of gastric solution prepared by mixing tap water with Simulated Gastric Fluid, SGF, comprising 3.1 g hydrochloric acid, 2 g sodium chloride, 3.2 g pepsin (from porcine gastric mucosa with 0.7 FIP-U/mg from Merck) and 1000 ml distillated water (Chellat F., Tabrizian M., Dumitriu S., Chomet E., Rivard C. H., Yahia L'Hocine in “Study of Biodegradation Behavior of Chitosan-Xanthan Microspheres in Simulated Physiological Media” J Biomed Mater Res (Appl Biomater) 53: 592-599, 2000) in a pre-established ratio, and a stirring bar. While stirring at 600 r.p.m. on a magnetic stirrer, 2.0 g of a PMSF sample was added, whereby gelation took place due to water absorption and swelling. This lead to a decrease in fluidity and disappearance of the eddy around the center of stirring. The time from the addition of the solid sample to the disappearance of the eddy was measured and shown as an index for the rate of liquid absorbency.

### Critical Stress

The critical stress, “τ_{c}”, in the context of the present invention, represents the stress that must be applied on a suspension material, gel, or solution to obtain the conditions

G′=G″

Tan δ=G″/G′=1

known as the “viscous-elastic transition” or as “gel point stress” which documents the flowability of the material (Schramm G. A. in “A Practical Approach to Rheology and Rheometry” Karlsruhe, Germany: Gebrueder HAAKE GmbH, pp 1718. 1994),

where:

G′—storage modulus, [kPa], is representative of the elastic properties of a material;
G″—loss modulus, [kPa], is representative of the viscous properties of a material; and
Tan δ—tangent of phase shift.

The critical stress in general is higher than the strength corresponding to “yield point”, which defines the point over which a material begins to flow. With heterogeneous materials, critical stress is a preferred measurement because experimental determination of yield point is difficult to achieve.

Critical stress τ_{c}, has been evaluated from Oscillation Stress Sweep rheological experiments using a RheoStress 1 rheometer from ThermoHaake with a cylindrical sensor system Z20 DIN, according to DIN 53019/ISO 3219. Critical stress values range from 0.5 Pa to 500 Pa, at constant frequency of 1 Hz at 37° C. About 8 grams of gel resulting from contact between PMSF, water, and SGF, is placed in the cup of the cylindrical sensor system. After the cup is fixed in the thermostat device of the rheometer, the rotor is placed in the gel. After 15 minutes of stabilizing the system at 37° C., the rheological test begins.

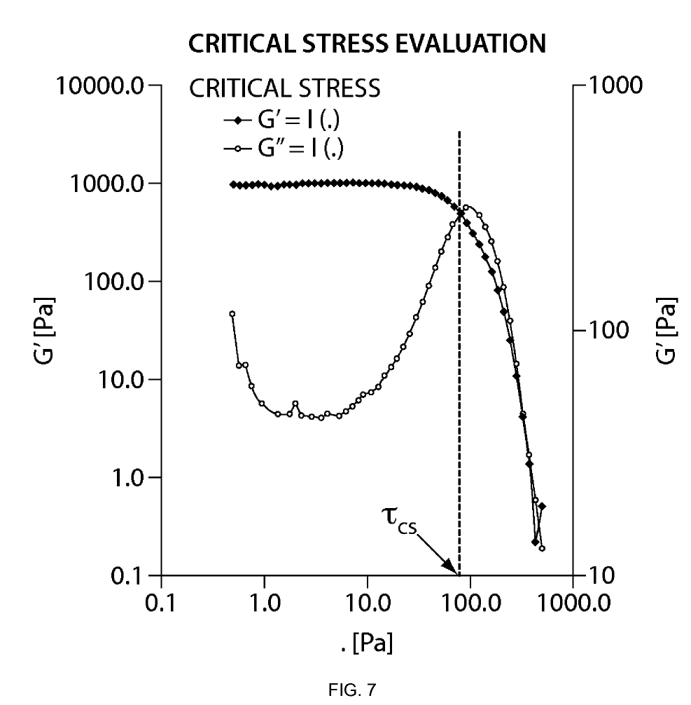

The experimental data was processed with the software RheoWinPro-Data Manager of ThermoHaake, using the sub program Crossover, to find the strength at which G′=G″. The value of critical stress, τ_{c}, was expressed as an average of three replicates. The graphical process of determining critical stress from the Oscillation Stress Sweep experimental data is depicted in FIG. 7.

### Simulated Gastric Swelling Test

PMSF of the present invention were evaluated by the “Simulated Gastric Swelling Test.”

The Simulated Gastric Swelling Test is an experiment in which the behavior of the PMSF from the moment it arrives in the stomach (start time t₁=tₘ=0 (min)), until evacuation through pyloric sphincter begins (stop time t₅=tₑₘₚ (min)) is monitored. The purpose of the test is to appreciate how PMSF simulates the behavior of normal food in the stomach, from entering to emptying, having as control:
[τ_{c}][_{[full]NF}], critical stress of normal food, at which is perceived the sensation of fullness; and
[τ_{c}][_{[empl]NF}], critical stress of normal chyme, when the emptying of the stomach begins.

Because experimental data that permits the correlation of rheological properties between the alimentary bolus formed from a regular meal and chyme aren't found, values for [τ_{c}]_{[full]NF}] and [τ_{c}]_{[emp]NF}] are established by an experiment referred to as “Simulated Gastric Behavior of normal food.” Simulated Gastric Behavior of normal food consists of the following:

The menu for a regular meal comprises
a “Big Mac” from McDonald's formed from 3 slices of bread, 2 slices of burger, leafs of salad, pickled cucumbers, and mayonnaise for a total weight of 200 g;
chips, for a total weight of 150 g
mineral water, “Mi Eden” (without carbon dioxide), 300 ml.
   Preparation of the “alimentary bolus” is done by manually cutting pieces of the Big Mac and chips, followed by mixing them together with mineral water in a kitchen blender for 10 minutes on minimum speed making a consistent paste similar to dough.
   Acidulation of alimentary bolus, simulation of contact with gastric juice in an empty stomach is realized by adding 50 ml of SGF over the paste from the blender, followed by homogenization over 1 minute.

The resulting mixture has been tested rheologically for critical stress (in conformity with the method presented above). The value [τ_{c}]_{[full]NF}=108.3 Pa was obtained.

Digestion of alimentary bolus and its transformation into chyme was carried out by transferring the acidulate paste from the blender to a laboratory planetary mixer where it was dosed continuously with 300 ml solution of SGF over 4 hours at 37° C. The amount of SGF added is based on the average value of gastric juice secretion, 75 ml/h. Digestion was subjectively limited to 4 hours based on the time interval that 10 subjects perceived the sensation of being full after eating a Big Mac. The resulting paste had diminished consistency versus its initial state. Critical stress was measured at [τ_{c}]_{[se]NF}=5.2 Pa using the critical stress test. Rheological values [τ_{c}]_{[full]NF} and [τ_{c}]_{[se]NF} can be modified versus the medical protocols adopted for the treatment of overweight and/or obesity.

In a laboratory planetary mixer (ARTISAN model MKSM 150 from KitchenAid, USA), a pre-determined quantity of PMSF, “mₑₓₚ” (grams), and a pre-determined volume of aqueous solution, “V_{Liq}” (ml) made up of a volume of water, “Vl_{w}”, and a volume of simulated gastric juice, “V_{SGF}” are moderately mixed (level 1, approximately 60 rpm) at 37° C. At the same time as mixing begins, supplemental SGF solution is added at a rate of 75 ml/hour which is represented as “V3ₜ” corresponding to dosing time “t”. Samples of gel are extracted from the mixture every 15 minutes over 6 hours. Samples of the gel are subjected to the following analysis:
critical stress at time t, “[τ_{c}]ₜ”, using 8 grams of mixture and applying the rheological evaluation methods described above;
degree of liquid absorbency at time t, “DLAₜ” expressed in percent (%), that comprises subjecting a 20 gram sample of the mixture to 500 mbar of pressure (in conformity with the method described above) and registering the liquid volume lost, “V4ₜ”, using the following formula

$${DLA}_{t} = \frac{[ ( {V}_{Liq} + V {3}_{t} ) - V {4}_{t} ⋆ \frac{( {V}_{Lig} + V {3}_{t} + {m}_{exp} )}{20} ]}{{V}_{Liq} + V {3}_{t}} ⋆ 100 , [ \% ]$$

dimension of gel particles represented by equivalent average diameter, “(Φ_{eq})ₜ”, evaluated from a sample of gel phase subjected to suction as above and from a minimum of 50 particles, by photography and computer image processing using Paint Shop Pro 8 and Excel from Microsoft.

Alternatively, the series of experimental data (τ_{c})ₜ, DLAₜ and (Φ_{eq})ₜ has been determined graphically as a function of time. From the graphs the following values have been determined:
fullness time, t_{full}, in minutes;
fullness critical stress, [τ_{c}]_{full}, in Pa;
dry gel time, t_{dry}, in minutes;
time of starting stomach emptying, tₑₘₚ, in minutes; and
critical stress for starting stomach emptying, [τ_{c}]ₛₑ, in Pa;
   that in the end was compared with the rheological values of normal food.

### Biodegradation Test

The goal of this test is to determine biodegradation capacity of “artificial chyme”, after its interaction with pancreatic juices. In the cup of the rheometer (Cylinder Sensor System, Z20 DIN RheoStress 1 from ThermoHaake), are placed 5 grams of artificial chyme that corresponds rheologically to evacuation conditions and which beforehand was subjected to suction (in conformity with the method described above) and 3 ml Simulated Intestinal Fluid, SIF. The SIF was prepared by dissolving 6.8 g of monobasic potassium phosphate in 250 mL of water. The solution was mixed and 190 mL of 0.2 N sodium hydroxide and 400 mL of water and 10 g of pancreatin (ACROS) were added. The pH was then adjusted with 0.2 N sodium hydroxide to 7.5±0.1 and the volume adjusted to 1 L with water (Chellat F., Tabrizian M., Dumitriu S., Chornet E., Rivard C. H., Yahia L'Hocine in “Study of Biodegradation Behavior of Chitosan-Xanthan Microspheres in Simulated Physiological Media” J Biomed Mater Res (Appl Biomater) 53: 592-599, 2000).

The cup of the cylinder sensor is fixed with thermostat bath at 37° C., and stirred with the rotor. After 15 minutes the Oscillation Time Sweep test begins. At a constant frequency of 1 Hz, at constant stress τ=1 Pa, and 37° C. for 2 hours (7200 sec), the values G′ and G″ are recorded. The experimental data is then processed with RheoWinPro-Data Manager software of ThermoHaake, using the subprogram Crossover, for determining the time at which G′=G″. This time is referred to as “biodegradation time”, t_{bio}, in minutes. The time t_{bio} shows that PMSF, as artificial chyme, suffers a process of enzymatic biodegradation from gel to a polymeric solution. Also, t_{bio} reflects sensitivity to enzymatic biodegradation of PMSF.

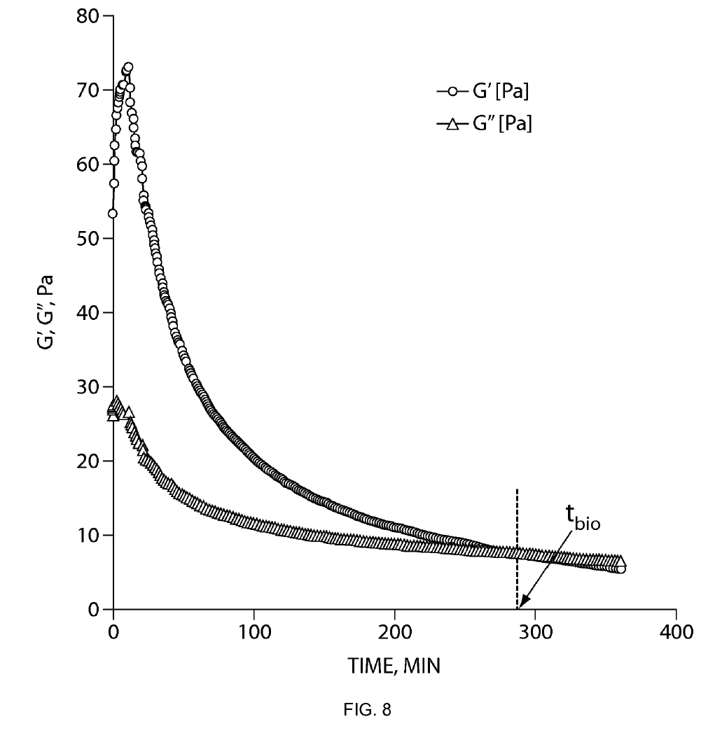

The graphical process for converting the rheological test data to t_{bio} is exemplified in FIG. 8.

### EXEMPLIFICATION

Referring to the Examples, the present invention is further explained below in more detail. However, these Examples are merely by way of illustration and not by way of limitation.

SMAC has been characterized from the point of view of maleic acid content and of viscosimetric average molecular weight, obtaining the results presented in Table 1.

TABLE 1
SMAC polymer characterization.

| | BOP for | Viscosimetric | Maleic acid |
|---|---|---|---|
| Name of | Synthesis | Molecular | in SMAC |
| polymer | [%] | weight Mᵥ [Da] | [mol %] |
| SMAC-1 | 0.15 | 1,980,000 | 0.495 |
| SMAC-2 | 0.3 | 1,615,000 | 0.488 |
| SMAC-3 | 0.55 | 1,050,000 | 0.49 |

The molecular weight, Mv, of SMAC has been determined using the viscosimetric method (Raju K. V. S, N., Yaseen M. “A new Equation for Estimating [q] from Single-Viscosity Measurement in Dilute Solution” J. Appl. Polym. Sci., 45, 677-681, 1992; Chee K. K. “A Critical Evaluation of the Single-Point Determination of Intrinsic Viscosity”, J. Appl. Polym. Sci., 34, 891-899, 1987 and Spiridon D., Panaitescu L., Ursu D., Uglea C. V., Popa I., Ottenbrite R. M. “Synthesis and Biocompatibility of Maleic Anhydride Copolymers: 1. Maleic Anhydride—Vinyl Acetate, Maleic Anhydride—Methyl Methacrylate and Maleic Anhydride—Styrene”, Polymer International, 43, 175-181, 1997), with tetrahydrofuran as solvent.

The monomeric composition of SMAC polymers was determined by potentiometric titration (Vilcu R., Ionescu Bujor, I., Olteanu M., Demetrescu I. “Thermal stability of copolymer acrylamide-maleic anhydride” J. Appl. Polym. Sci., 33: 2431-2437, 1987).

### Examples 1-5

These examples present methods of preparing finished PMSF products PMSF-1, PMSF-2, PMSF-3, PMSF-4, and PMSF-5, using the same operation mode, but with the different parameters specified in Table 2. The operation mode for obtaining the finished products comprises the following.

Quantities of raw materials necessary for preparation were: m_{A} grams of synthetic polymer; m_{B} grams of gelatin (from porcine skin, SIGMA Catalog number 9000-70-8); m_{C} grams of alkaline agents (NaOH or NH₄OH, from ACROS) and m_{w} grams of distilled water (10 μs conductivity) used to prepare the polymeric composite in solution state [ABC-sol] with a solids content, cₛ, of 25%.

SOL-C was prepared by dissolving m_{C} grams of alkaline agents in 100 g of distilled water in a 150 ml beaker. SOL-B was prepared by placing m_{B} grams of gelatin in a 150 mL beaker with m_{w}-100/2 g of distilled water. The gel was allowed to swell over 24 hours at room temperature. The resulting gel was melted at 50° C. and the rest of the water was added while stirring to obtain SOL-B with a content of solid substance, cₛ, of 25%.

In a laboratory kneader (MKD 0.6-H60 IKA Catalog, Laboratory Technology) having a working volume of 500 ml and equipped with a heating-cooling mantle, m_{A} grams of synthetic polymer and SOL-C were added and mixed at 60° C. for 2 hours. SOL-B pre-heated to 50° C. was added and mixing continued at the same realized for 3 hours. [ABC-sol] was obtained as a mixture with a consistency similar to a polymer melt.

The viscous mass of [ABC-sol] was cooled to 30° C., evacuated from the kneader and was extruded from a meat chopper (Food Grinder of ARTISAN model MKSM 150 from KitchenAid, USA) equipped with a stainless steel perforated plate with 5 mm holes. The 12 mm cylindrical pieces of material were discharged onto a metallic framework covered with a stainless steel wire net with 250 micron holes. The frame with the material pieces was placed in an oven with circulating hot air (Laboratory Air Circulation Oven HERAEUS model UT 12, from KENDRO Laboratory Products, Germany) to eliminate excess water by evaporation. The material drying occurred in hot air at 65° C. for 6 hours yielding m_{solid} grams of solid material with a humidity content, w_{dry} as a %. The dried material was ground in a cone mill (cone mill from MAZZER Luigi slr, Italy). The grounded material was separated by sieving with vibrating sieves (Vibratory Sieve Shaker, model Analysette 3, from FRITSCH, Germany) in two solid fractions: m1_{solid} grams with d_{eq}=0.2-0.8 mm or d_{eq}=0.5-1.0 that represent [ABC-dry] and m2_{solid} grams with granulometric characteristics of d_{eq} less than 0.2 mm which represent [ABC-rec]. The grading fraction [ABC-rec] was collected for reprocessing.

The granular mass [ABC-dry] is uniformly distributed on a stainless steel tray and placed in a laboratory oven (the same used for drying) pre-heated at T₁° C. and maintained for t₁ hours. Lastly, the granular mass was taken out and cooled to room temperature yielding PMSF as finished product which was collected in polyethylene boxes of 100 g each and hermetically sealed for 24 hours.

TABLE 2
Preparation parameters and properties for Examples 1-5.

| | Samples from Examples 1-5 | | | | |
|---|---|---|---|---|---|
| Preparation Parameters | PMSF-1 | PMSF-2 | PMSF-3 | PMSF-4 | PMSF-5 |

Raw Material and Chemical Compositions of PMFS samples

| | | | | | |
|---|---|---|---|---|---|
| SMAC type | SMAC-2 | SMAC-2 | SMAC-2 | SMAC-2 | SMAC-2 |
| M_{A}, [g] | 45 | 40 | 35 | 45 | 35 |
| Gelatin type | Type A | Type A | Type A | Type A | Type A |
| | (bovine) | (bovine) | (bovine) | (bovine) | (bovine) |
| Bloom index | 175 | 175 | 175 | 175 | 175 |
| M_{B}, [g] | 5 | 10 | 15 | 5 | 15 |
| A:B | 90:10 | 80:20 | 70:30 | 90:10 | 70:30 |
| C⁽⁺⁾, type | Na⁽⁺⁾ | NH₄⁽⁺⁾ | Na⁽⁺⁾ | Na⁽⁺⁾ | Na⁽⁺⁾ |
| C⁽⁺⁾, mol/g (A + B) | 0.006 | 0.006 | 0.005 | 0.007 | 0.007 |
| m_{c}, [g] | 12 | 10.5 | 10 | 14 | 14 |
| m_{w}, [g] | 186 | 181.5 | 180 | 192 | 192 |

Technological Parameters

| | | | | | |
|---|---|---|---|---|---|
| [ABC-Sol], cₛ, [%] | 25 | 25 | 25 | 25 | 25 |
| Drying Temparature, [° C.] | 65 | 65 | 65 | 65 | 65 |
| Drying time, [hours] | 6 | 6 | 6 | 6 | 6 |
| Thermal Cross-linking | 110 | 125 | 105 | 110 | 115 |
| Temperature, T₁, [° C.] | | | | | |
| Thermal Cross-linking | 90 | 60 | 180 | 120 | 60 |
| Time, t₁, [min] | | | | | |

Result of Preparation

| | | | | | |
|---|---|---|---|---|---|
| ml_{solid}, [ABC-dry], [g] | 50.8 | 51.4 | 49.8 | 53.1 | 53.7 |
| W_{dry} [%] | 7.2 | 8.03 | 7.76 | 7.41 | 8.12 |
| Particles dimension, [mm] | 0.2-0.8 | 0.5-1.0 | 0.2-0.8 | 0.2-0.8 | 0.2-0.8 |
| (Φ_{eq})_{dry}, [mm] | 0.568 | 0.843 | 0.496 | 0.524 | 0.612 |
| m2_{solid}, [ABC-rec], [g] | 11.2 | 9.1 | 10.2 | 10.8 | 10.3 |
| PMSF, [g] | 50.8 | 51.4 | 49.8_{—} | 53.1 | 53.7 |
| W, [%] | 7.24 | 8.03 | 7.76 | I 7.41 | 8.12 |

The finished products PMSF-1, PMSF-2, PMSF-3, PMSF-4, and PMSF-5 were analyzed according to the methods presented under “Test Procedures.” The testing conditions and the results are presented in Table 3.

TABLE 3
Finished product characterization of samples from Examples 1-5.

| | Samples from Examples 1-5 | | | | |
|---|---|---|---|---|---|
| Properties | PMSF-1 | PMSF-2 | PMSF-3 | PMSF-4 | PMSF-5 |

General Properties

| | | | | | |
|---|---|---|---|---|---|
| FADW, [g/g] | 336 | 306 | 278 | 351 | 322 |
| E, [kPa] | 1.15 | 0.84 | 1.58 | 1.26 | 2.31 |
| AcBC, [mEq [HCI/g] | 6.35 | 6.59 | 5.77 | 6.28 | 7.83 |

The conditions of Simulated Gastric Swelling Tests

| | | | | | |
|---|---|---|---|---|---|
| mₑₓₚ, [g] | 6 | 8 | 8 | 4 | 6 |
| V_{Liq}, [ml] | 450 | 360 | 380 | 250 | 450 |
| V1w, [ml] | 400 | 300 | 300 | 200 | 400 |
| V2_{SGF}, [ml] | 50 | 60 | 80 | 50 | 50 |
| V3_{SGF}, [ml/hour] | 75 | 75 | 75 | 75 | 75 |

The Properties of PMSF correlated with Full-stomach Principle

| | | | | | |
|---|---|---|---|---|---|
| t_{gel}, [sec] | 127 | 131 | 169 | 104 | 152 |
| t_{full}, [min] | 10 | 20 | 30 | 10 | 20 |
| [τ_{c}]_{full}, Pa | 108.3 | 108.3 | 108.3 | 108.3 | 108.3 |
| t_{dry}, [min] | 75 | 60 | 90 | 105 | 75 |
| tₛₑ, [min] | 255 | 150 | 315 | 270 | 330 |
| [τ_{C}]ₛₑ Pa | 5.6 | 1.23 | 3.44 | 6.22 | 2.58 |
| (Φ)_{eq) full}, [mm] | 4.28 | 6.33 | 5.11 | 5.07 | 6.44 |
| (Φ)_{eq )se}, [mm] | 2.08 | 1.56 | 0.783 | 1.825 | 1.663 |
| Tₑₘₚ, [min] | 315 | 255 | 345 | 300 | 360 |
| T_{bio}, [min] | 66 | 52 | 45 | 98 | 39 |

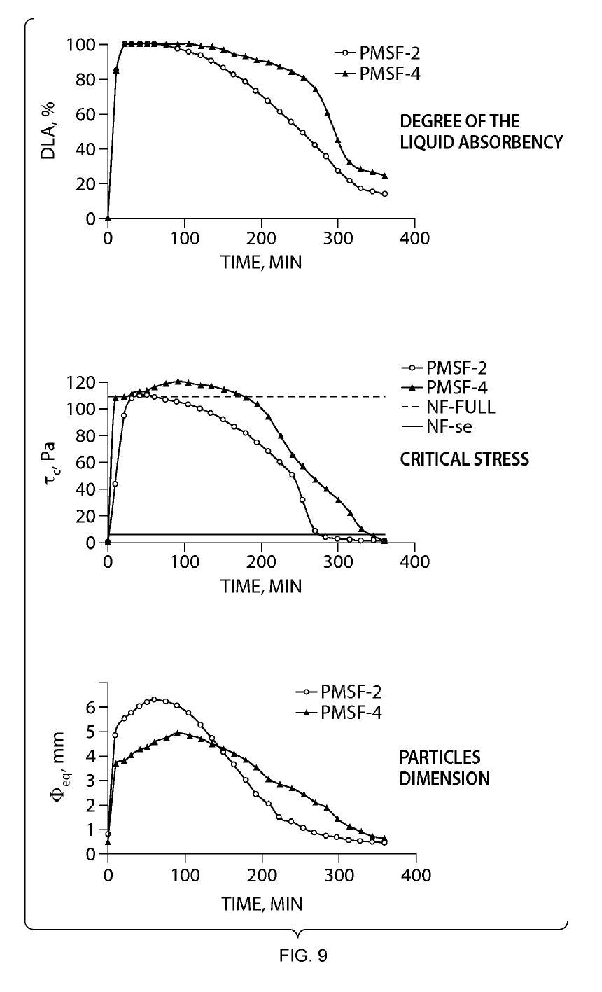

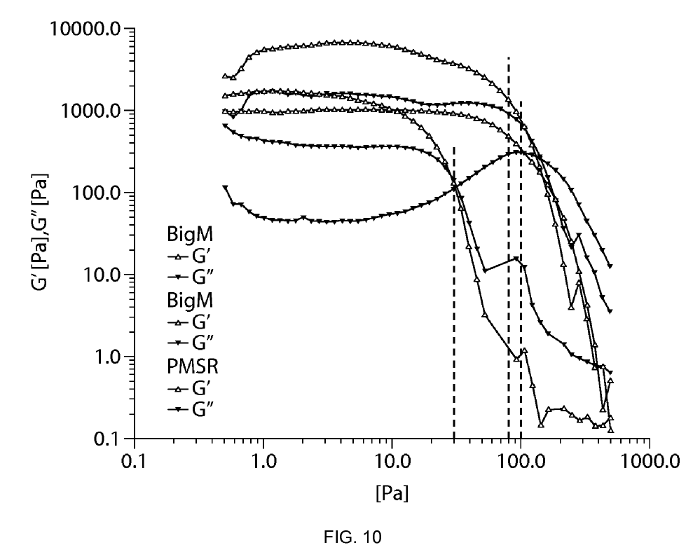

Evaluation of the properties associated with Simulated Gastric Swelling for PMSF-1 and PMSF-5 and how they vary with time is depicted for (DLA)ₜ, [τ_{c}]ₜ, and (Φ)_{(eq)t} presented in FIGS. 9 and 10.

### Examples 6-10

These examples present methods of preparing finished PMSF products PMSF-6, PMSF-7, PMSF-8, PMSF-9, and PMSF-10, using the same operation mode, but with the different parameters specified in Table 4. The operation mode for obtaining the finished products comprises the following.

Quantities of raw materials necessary for preparation were: m_{A1} grams of synthetic polymer; m_{B1} grams of gelatin (from porcine skin from SIGMA Catalog number 9000-70-8); m_{C}, grams of alkaline agents (NaOH from ACROS) and m_{W1}, grams of distilled water (10 μS conductivity) used to prepare the polymeric composite in solution state [ABC-sol].

To prepare SOL-C, m_{c1} grams of alkaline agents were dissolved in 100 g of distilled water by simple mixing in a 150 ml beaker. To prepare SOL-B, m_{B1} grams of gelatin were placed in a 150 ml beaker. (m_{w}-100)/2 g of distilled water were added and the gelatin was allowed to swell for 24 hours at room temperature. The resulting gel was melted at 50° C. and addition of the remaining water over the resulting solution with stirring forms SOL-B with a solid substance content of cₛ.

In a laboratory kneader (MKD 0.6-H60 IKA Catalog, Laboratory Technology) having a working volume of 500 ml and equipped with a heating-cooling mantle, M_{A} grams of synthetic polymer and SOL-C were mixed at 60° C. for 2 hours. SOL-B pre-heated to 50° C. is added to the mixture. The mixing continued at the temperature realized for 3 hours. [ABC-sol] is obtained with a consistency similar to a polymer melt.

The viscous mass of [ABC-sol] was cooled to 30° C. and removed from the kneader by extrusion through a meat chopper (Food Grinder of ARTISAN model MKSM 150 from KitchenAid, USA) equipped with a stainless steel perforated plate with 5 mm holes. The 12 mm cylindrical pieces of material are discharged onto a metallic frame covered with a stainless steel wire net with 250 micron holes. The frame is introduced in an oven with circulating hot air (Laboratory Air Circulation Oven HERAEUS model UT 12, from KENDRO Laboratory Products, Germany) to eliminate the water excess by evaporation. Drying occurred at 65° C. during 6 hours yielding m_{solid} grams of solid material with a humidity content W_{dry}, %. The dried material resulted was ground in a cone mill (cone mill, from MAZZER Luigi slr, Italy), after which, the material was separated by sieving with vibrating sieves (Vibratory Sieve Shaker, model Analysette 3, from FRITSCH, Germany) as two solid fractions: m₁ solid grams with d_{eq}=0.2-1.5 mm, represented as [ABC-dry] and other m₂ solid grams with granulometric characteristics d_{eq} less than 0.2 mm, represented as [ABC-rec]. The grading fraction [ABC-rec] was collected for re-processing.

[ABC-dry] was uniformly distributed on a stainless steel tray and placed in a laboratory oven with air pre-heated at T₁° C. where it was maintained for t₁ hours. The granular mass was taken out of the oven and cooled to 40-45° C. The resulting PMSF-gross grams was collected in 50 g polyethylene boxes with hermetical seals and stored for 24 hours at a median temperature of 24° C.

The PMSF-gross was placed in the same laboratory oven as above with pre-heated air at T₂° C. and maintained for T₂ minutes. Lastly, the granular mass was removed from the oven and cooled to 40-45° C. yielding the PMSF finished product with a humidity content W, %. The PMSF finished product was collected in a polyethylene box with hermetic seals and was stocked at an ambient temperature for maturation of the product for 24 hours.

TABLE 4
Preparation Parameters and Properties for examples 6-10.

| | Samples from examples 6-10 | | | | |
|---|---|---|---|---|---|
| Preparation Parameters | PMSF-6 | PMSF-7 | PMSF-8 | PMSF-9 | PMSF-10 |

Raw Material and Chemical Compositions of PMFS sam,les

| | | | | | |
|---|---|---|---|---|---|
| SMAC type | SMAC-2 | SMAC-2 | SMAC-2 | SMAC-1 | SMAC-3 |
| M_{A}, [g] | 45 | 48 | 48 | 42.5 | 52.5 |
| Gelatin type | Type A | Type A | Type A | Type A | Type A |
| | (bovine) | (bovine) | (bovine) | (pork) | (pork) |
| Bloom index | 175 | 100 | 300 | 225 | 225 |
| M_{B}, [g] | 5 | 12 | 12 | 7.5 | 17.5 |
| A:B | 90:10 | 80:20 | 80:20 | 85 15 | 75:25 |
| C⁽⁺⁾, type | Na⁽⁺⁾ | NH⁽⁺⁾ | Na⁽⁺⁾ | Na⁽⁺⁾ | Na⁽⁺⁾ |
| C⁽⁺⁾, mol/g (A + B) | 0.006 | 0.005 | 0.0055 | 0.0065 | 0.006 |
| m_{c}, [g] | 12 | 10.5 | 13.2 | 13 | 16.8 |
| m_{w}, [g] | 186 | 282 | 414.8 | 357 | 161 |

Technological Parameters

| | | | | | |
|---|---|---|---|---|---|
| [ABC-Sol], cₛ, [%] | 25 | 20 | 15 | 15 | 35 |
| Drying Temparature, [° C.] | 65 | 65 | 65 | 50 | 90 |
| Drying time, [hours] | 6 | 6 | 6 | 8 | 4 |
| Thermal Cross-linking | 110 | 125 | 105 | 110 | 115 |
| Termperature, T₁, [° C.] | | | | | |
| Thermal Cross-linking | 90 | 60 | 180 | 120 | 60 |
| Time, t₁, [min] | | | | | |
| Thermal surface | 130 | 135 | 130 | 125 | 135 |
| consolidation | | | | | |
| Time, T₂, [min] | | | | | |
| Thermal surface | 15 | 20 | 10 | 20 | 15 |
| consolidation | | | | | |
| Time, t₂, [min] | | | | | |

Result of Preparation

| | | | | | |
|---|---|---|---|---|---|
| ml_{solid}, [ABC-dry], [g] | 50.8 | 59.2 | 59.3 | 53.5 | 73.1 |
| W_{dry} [%] | 7.2 | 6.88 | 8.15 | 6.92 | 7.27 |
| Particles dimension, [mm] | 0.2-0.8 | 0.5-1.0 | 0.2-0.8 | 0.8 | 0.2-0.8 |
| (Φ_{eq})_{dry}, [mm] | 0.568 | 0.744 | 0.619 | 1.283 | 0.583 |
| m_{2solid} [ABC-rec], [g] | 11.2 | 11.28 | 13.9 | 9.45 | 13.88 |
| PMSF, [g] | 49.6 | 58.7 | 58.8 | 53.1 | 72.6 |
| W, [%] | 5.87 | 5.11 | 5.23 | 5.06 | 5.41 |

The finished products PMSF-6, PMSF-7, PMSF-8, PMSF-9, and PMSF-10 have been analyzed in conformity with the methods presented under “Test Procedures.” The testing conditions and results obtained are presented in Table 5.

TABLE 5
Finished product characterization from examples 6-10.

| | Samples from examples 1-5 | | | | |
|---|---|---|---|---|---|
| Properties | PMSF-6 | PMSF-7 | PMSF-8 | PMSF-9 | PMSF-10 |

General Properties

| | | | | | |
|---|---|---|---|---|---|
| FADW, [g/g] | 251 | 283 | 245 | 278 | 306 |
| E, [kPa] | 3.22 | 4.58 | 5.39 | 7.47 | 2.07 |
| AcBC, [mEq [HCI/g] | 6.35 | 5.21 | 5.83 | 6.84 | 6.25 |

The conditions of Simulated Gastric Swelling Tests

| | | | | | |
|---|---|---|---|---|---|
| mₑₓₚ, [g] | 10 | 15 | 12 | 8 | 14 |
| V_{Liq}, [ml] | 450 | 400 | 500 | 450 | 450 |
| V1W, [ml] | 400 | 350 | 400 | 400 | 400 |
| V2_{SGF}, [ml] | 50 | 50 | 100 | 50 | 50 |
| V3_{SGF}, [ml/hour] | 75 | 75 | 75 | 75 | 75 |

The Properties of PMSF, correlated with “Full-stomach Principle”

| | | | | | |
|---|---|---|---|---|---|
| t_{gel}, [sec] | 103 | 85 | 158 | 172 | 95 |
| t_{full}, [min] | 10 | 10 | 20 | 30 | 10 |
| [τ_{c}]_{full}, Pa | 108.3 | 108.3 | 108.3 | 108.3 | 108.3 |
| t_{dry}, [min] | 90 | 60 | 105 | 135 | 75 |
| tₛₑ, [min] | 285 | 165 | 225 | 300 | 265 |
| [τ_{C}]ₛₑ Pa | 7.32 | 4.52 | 1.73 | 9.41 | 2.37 |
| (Φ)_{eq) full}, [mm] | 3.55 | 8.238 | 6.21 | 10.217 | 5.83 |
| (Φ)_{eq )se}, [mm] | 2.27 | 1.56 | 2.083 | 3.691* | 1.944 |
| tₑₘₚ, [min] | 345 | 315 | 330 | >360 | 285 |
| t_{bio}, [min] | 83 | 91 | 98 | 117 | 107 |

*Particles of “artificial chyme” are bigger than the dimensions of pyloric sphincter which means the stationary time in the stomach is extended and it is possible to introduce a new quantity of water to determine a supplementary acidulation and implicitly decreasing of particles dimension.

The examples presented are not restrictive, the PMSF finished products can be adapted to each type of medical protocol for treatment of overweight and/or obesity, both from the point of view of the “Full-stomach principle” and in association with other treatment strategies.

### REFERENCES CITED

U.S. Pat. Nos. 3,046,988; 3,507,952; 3,859,942; 3,926,891; 3,935,099; 3,959,569; 3,980,663; 3,983,095; 3,997,484; 4,076,663; 4,090,013; 4,117,184; 4,124,748; 4,133,315; 4,190,562; 4,246,893; 4,207,890; 4,264,155; 4,389,513; 4,416,267; 4,434,153; 4,485,805; 4,525,527; 4,570,629; 4,610,678; 4,654,039; 4,666,983; 4,735,804; 4,739,758; 4,767,627; 4,784,002; 4,808,637; 4,833,222; 4,837,285; 4,855,179; 4,899,747; 4,950,485; 4,959,341; 4,971,954; 5,063,073; 5,075,344; 5,118,719; 5,153,174; 5,284,936; 5,336,486; 5,352,448; 5,405,616; 5,408,019; 5,453,323; 5,489,261; 5,567,478; 5,629,377; 5,654,028; 5,674,495; 5,676,967; 5,712,316; 5,750,585; 5,760,082; 5,795,895; 5,847,013; 5,847,03; 5,859,077; 5,863,984; 5,869,080; 5,948,429; 6,018,033; 6,020,324; 6,071,544; 6,103,269; 6,107,432; 6,168,762; 6,271,278; 6,309,853; 6,322,826; 6,403,657; 6,426,077; 6,468,962; 6,468,988; 6,472,002; 6,475,530; 6,507,109; 6,833,488; and RE33997
Foreign Patent Documents: WO 04156343 and WO 05184724.

### OTHER REFERENCES

1. Chen Jun, Blevins W. E., Park Haesun, Park Kinam in “Gastric retention properties of superporous hydrogel composite,” J. Controlled Release, 64, 39-51, 2000.
2. Freita Jr, R. A., in “Nanomedicine”, Volume 1 Landes Bioscience, Georgetown, Tex., 1999.
3. Johnson L. R. in “Physiology of the Gastrointestinal Tract”, Raven Press, New York, 1981.
4. Nielsen, L. E. in “Mechanical Properties of Polymers Composites”; Marcel Dekker: New York, 1974.
5. Schramm G A. in “A Practical Approach to Rheology and Rheometry”. Karlsruhe, Germany: Gebrueder HAAKE GmbH, pp 17-18. 1994.
6. Morita R., Honda R., Takahashi Y., “Development of oral controlled preparation, a PVA swelling controlled release system, SCRS. I. Design of SCRS and its release controlling factor”, J. Controlled Release, 63,297-304, 2000.
7. Shalaby W. S. W., Blevins W. E., Park K., “In vitro and in vivo studies of enzyme-digestible hydrogels for oral drug delivery”, J. Controlled Release, 19, 289-296, 1992.
8. Podual K., Doyle F. J., Pappas N. A., “Dynamic behavior of glucose oxidaze-containing microparticles of poly(ethylene glycol)-grafted cationic hydrogels in an environment of changing pH”, Biomaterials 21, 14391450, 2000.
9. Deshpande A. A., Shah N. H., Rhodes C. T., Malick W., “Development of a novel controlled-release system for gastric retention”, Pharm, Res., 14, 815-819, 1997.
10. Hang Y., Leobandung W., Foss A., Peppas N. A. “Molecular aspects of muco- and bioadhesion: Treated structures and site-specific surfaces”, J. Controlled Release, 65, 63-71, 2000.
11. Black J., “Biological Performance of Materials: Fundamentals of Biocompatibility”, 2d ed. M. Dekker, N.Y., 1992.
12. Breitkreutz, J. in “Leakage of enteric (Eudragit L)-coated dosage forms in simulated gastric juice in the presence of poly(ethylene glycol)”, Journal of Controlled Release 67: 79-88, 2000.
13. Sethi, N., R. K et al. in—“Safety evaluation of a male injectable antifertility agent, styrene maleic anhydride copolymer, in rats”, Contraception 39:217-226, 1989.
14. Lohiya N. K et al. in “Repeated vas occlusion and non-invasive reversal with styrene maleic anhydride for male contraception in langur monkeys”, Int J Androl; 23: 36-42, 2000.
15. Ottenbrite, R. M. in 11 Antitumor activity of polycarboxylic acid polymers”, J. Macromol. Sci.-Chem., A22(5-7), 819-832, 1985.
16. Spiridon D., Panaitescu L., Ursu D., Uglea C. V., Popa I., Ottenbrite R. M. in “Synthesis and Biocompatibility of Maleic Anhydryde Copolymers 1. Maleic Anhydride—Vinyl Acetate, Maleic Anhydride—Methyl Methacrylate and Maleic Anhydride—Styrene”, Polymer International, 43 175-181, 1997.
17. Hoffman A. S., Daly C. H., “Boplogy of Collagen”, Viidik Vunst J. Eds., Academic Press New York, 1980; Ward A. G., Courts A., “The Science and Technology of Gelatin,” Academic Press N.Y., 1977.
18. Bo J., “Study on PVA Hydrogel Crosslinked by Epiclorohydrin”, J. Appl. Polym. Sci., 46, 783-786, 1992.
19. Scotchford C. A., Cascone. G. D., Ownes S., Gusti P., “Osteoblast responses of collagen-PVA bioartificial polymers in vitro: the effects of cross-linking method and collagen content”, Biomaterials 19, 1-11, 1998.
20. Giunchedi P., Genta I., Conti B., Muzzarelli R. A. A., Conti B., Biomaterials 19, 157-161, 1998.
21. Raju K. V. S. N., Yaseen M.—“A new Equation for Estimating [0] from Single-Viscosity Measurement in Dilute Solution” J. Appl. Polym. Sci., 45, 677-681, 1992.
22. Chee K K.—“A Critical Evaluation of the Single-Point Determination of Intrinsic Viscosity”, J. Appl. Polym. Sci., 34, 891-899, 1987.
23. Vilcu, R., Tonescu Bujor, I., Olteanu M., Demetrescu I., in “Thermal stability of copolymer acrylamide-maleic anhydride”, J. Appl. Polym. Sci., 33: 2431-2437, 1987.
24. Gaylord N. G. “Copolymers, Polyblends and Composites” Adv chem. 142, 76, 1975.
25. Paul D. R., Newman S. “Polymer Blends”, Academic Press, New York, 1978.
26. Manson J. A., Sperling L. H.—“Polymer Blends and Composites”, Plenum Press, N.Y., 1976.

1-3. (canceled)

4. A swellable polymeric material represented by the formula:
[(AB)⁽⁻⁾C⁽⁺⁾]W
wherein,
A represents a carboxylic containing copolymer;
B represents a biopolymer;
C represents a counterion; and
W represents water bound to the polymer.

5. The swellable polymeric material of claim 4, wherein A comprises co-monomers M1 and M2 in ratio of 20:80 to 80:20.

6. (canceled)

7. The swellable polymeric material of claim 5, wherein M1 comprises co-monomers maleic anhydride and maleic acid.

8. The swellable polymeric material of claim 5, wherein M1 comprises (a) co-monomers itaconic anhydride and itaconic acid (b) co-monomers citraconic anhydride and citraconic acid; or (c) co-monomers 2-octenylsuccinic anhydride and 2-octenylsuccinic acid.

9. (canceled)

10. (canceled)

11. The swellable polymeric material of claim 5, wherein M2 comprises an olefin.

12. The swellable polymeric material of claim 5, wherein M2 comprises a monoolefin.

13. The swellable polymeric material of claim 5, wherein M2 comprises ethylene, propene, isobutylene, styrene, alpha-methylstyrene, alkylated styrenes, ethylstyrene, tertbutylstyrene, vinyl-toluene, vinyl esters of saturated C₁-C₄-carboxylic acids, vinyl formate, vinyl acetate, vinyl propionate, alkyl vinyl ethers, ethyl vinyl ether, butyl vinyl ether, acrylate, methacrylate esters, 2-ethylhexyl acrylate, n-butyl acrylate, isobutyl acrylate, t-butyl acrylate, hexyl acrylate, n-butyl methacrylate, lauryl methacrylate, isodecyl methacrylate, conjugated diolefins, butadiene, isoprene, piperylene, allenes, allene, methyl allene, chloroallene, olefin halides, vinyl chloride, vinyl fluoride, polyfluoro-olefins, esters of monoethylenically unsaturated C₃-C₆-carboxylic acids, esters of monohydric C₁-C₈— alcohols and acrylic acid, esters of monohydric C₁-C₈-alcohols and methacrylic acid, esters of monohydric C₁-C₈— alcohols and maleic acid, monoesters of maleic acid, monomethyl maleate, 2-hydroxyethyl acrylate, hydroxypropyl acrylate, hydroxybutyl acrylate, hydroxyethyl methacrylate, hydroxypropyl methacrylate, hydroxybutyl methacrylate, N-vinyllactams, N-vinylpyrrolidone, N-vinylcaprolactam, acrylic and methacrylic esters of alkoxylated monohydric saturated alcohols, vinyl pyridine, vinyl morpholine, N-vinylformamide, dialkyldiallylammonium halides, dimethyldiallylammonium chloride, diethyldiallylammonium chloride, allylpiperidinium bromide, N-vinylimidazoles, N-vinylimidazole, 1-vinyl-2-methylimidazole, N-vinylimidazolines, N-vinylimidazoline, 1-vinyl-2-methylimidazoline, 1-vinyl-2-ethylimidazoline, 1-vinyl-2-propylimidazoline, acrylic acid, methacrylic acid, acrylamide, methacrylamide or acrylonitrile.

14. The swellable polymeric material of claim 5, wherein M2 comprises styrene.

15. (canceled)

16. (canceled)

17. The swellable polymeric material of claim 5, wherein M1 comprises co-monomers maleic anhydride and maleic acid, and M2 comprises styrene.

18. The swellable polymeric material of claim 4, wherein B comprises a protein, soybean protein, collagen, collagenic biopolymers, gelatin, collagen hydrolysates, or albumin casein.

19. The swellable polymeric material of claim 4, wherein B is a gelatin or carbohydrate.

20. (canceled)

21. (canceled)

22. The swellable polymeric material of claim 4, wherein B has a Bloom Index not less than 20 and not higher than 500 bloom.

23. (canceled)

24. The swellable polymeric material of claim 4, wherein B has an isoelectric point (IP) not less than 3.5 and not greater than 9.5.

25. (canceled)

26. The swellable polymeric material of claim 4, wherein the ratio of A:B is from 95:5 to 55:45 by weight.

27. (canceled)

28. The swellable polymeric material of claim 4, wherein the ratio of A:B is 90:10, 85:15, 80:20, or 75:25 by weight.

29. The swellable polymeric material of claim 4, wherein C is an inorganic cation.

30. The swellable polymeric material of claim 4, wherein C is Li⁽⁺⁾, Na⁽⁺⁾, K⁽⁺⁾, or NH₄⁽⁺⁾.

31. (canceled)

32. The swellable polymeric material of claim 4, wherein the molar content of C⁽⁺⁾, expressed in mol/gram of (A+B), is not less than 0.002 mol/g and not greater than 0.004 mol/g.

33. (canceled)

34. The swellable polymeric material of claim 5, wherein M1 comprises co-monomers maleic anhydride and maleic acid, M2 comprises styrene, B is a gelatin, C is Na⁽⁺⁾ or NH₄⁽⁺⁾.

35. The swellable polymeric material of claim 34, wherein the ratio of A:B is from 95:5 to 55:45 by weight.

36. (canceled)

37. (canceled)

38. The swellable polymeric material of claim 1, wherein the polymeric material has a humidity content not less than 1% and not greater than 15% by weight.

39. (canceled)

40. The swellable polymeric material of claim 1, wherein the viscozimetric average molecular mass, My, is not less than 100,000 and not greater than 1,000,000 evaluated from intrinsic viscosity, [η], in tetrahydrofuran at 25° C.

41. (canceled)

42. The swellable polymeric material of claim 1, wherein the free absorbency for distillated water, FADW, at 37° C. after 24 hours of contact with water is not less than 200 g/g.

43. (canceled)

44. The swellable polymeric material of claim 1, wherein the acid binding capacity, ABC, in mEq HCl/g of polymeric material, is not less than 0.002 mEq HCl/g.

45. (canceled)

46. The swellable polymeric material of claim 1, wherein the swelling phenomenon occurs not more than 30 minutes after oral administration in a subject.

47. (canceled)

48. (canceled)

49. The swellable polymeric material of claim 1, wherein the time from oral administration of the swellable polymeric material to the perceived sensation of fullness in a subject is not greater than 30 minutes.

50. (canceled)

51. The swellable polymeric material of claim 1, wherein the time from oral administration of the swellable polymeric material to a subject to the start of stomach emptying is not less than 50 minutes.

52. The swellable polymeric material of claim 1, wherein the time from oral administration of the swellable polymeric material to a subject to the start of stomach emptying is not greater than 300 minutes.

53-55. (canceled)

56. The swellable polymeric material of claim 1, wherein the swellable polymeric material has the same rheological properties as ground food.

57. A composition comprising the swellable polymeric material of claim 1.

58. The composition of claim 57, further comprising a pharmaceutical carrier.

59. The composition of claim 57, in the form of a tablet, capsule, pill, or elixir.

60. A method of treating being over weight or obesity comprising administering to a subject in need thereof an effective amount of the swellable polymeric material of claim 1.

61. The method of claim 60, further comprising administering another form of treatment.

62. The method of claim 61, wherein the treatment is gene therapy, surgical interventions, or administering an appetite suppressant.

63. A method of inducing the feeling of satiety in a subject comprising administering to a subject in need thereof an effective amount of the swellable polymeric material of claim 1.

64. The method of claim 60, wherein the swellable polymeric material takes the place of a meal.

65. The method of claim 60, wherein the amount of swellable polymeric material is not less than 2 grams and not more than 20 grams.

66. (canceled)

67. The method of claim 60, wherein the swellable polymeric material is administered with water.

68. The method of claim 67, wherein the amount of water is not less than 100 ml and not greater than 600 ml.

69. (canceled)

70. A method of preparing a swellable polymeric material capable of inducing the sensation of satiety upon ingestion comprising:
a) preparing an aqueous mixture of a synthetic copolymer comprising carboxylic groups;
b) preparing an aqueous solution of an inorganic salt;
c) preparing an aqueous mixture of a biopolymer;
d) mixing the synthetic polymer mixture from step a) with the inorganic salt solution from step b) to form synthetic polymer-inorganic salt mixture;
e) adding the biopolymer mixture from step c) to the synthetic polymer-inorganic salt mixture of step d) to form an aqueous mixture of the polymeric material;
f) drying the polymeric material from step e); and
g) thermally crosslinking the polymeric material of step f) to form the swellable polymeric material.

71. The method of claim 70, wherein the aqueous mixture of the synthetic copolymer and aqueous solution of the inorganic salt are mixed at a temperature not less than 20° C. and not greater than 90° C.

72-74. (canceled)

75. The method of claim 70, wherein the aqueous mixture of the biopolymer is preheated to about 50° C.

76. The method of claim 70, wherein the synthetic polymer-inorganic salt mixture and biopolymer mixture are mixed at about 50° C.

77. The method of claim 70, wherein the synthetic polymer-inorganic salt mixture and biopolymer mixture are mixed for not less than 1 hour and not more than 4 hours.

78-80. (canceled)

81. The method of claim 70, wherein the polymeric material after drying has a humidity content of 5-10% by weight.

82. The method of claim 70, wherein the polymeric material is thermally crosslinked at a temperature not less than 100° C. and not greater than 130° C.

83. (canceled)

84. The method of claim 70, wherein the polymeric material is thermally crosslinked for not less than 30 minutes and not greater than 4 hours.

85-87. (canceled)
